(19) United States
(12) Patent Application Publication
(10) Pub. No.: US 2025/0271530 A1
(43) Pub. Date: Aug. 28, 2025

(54) SYSTEM AND METHOD OF MAGNETIC RESONANCE IMAGING FOR STUDYING TISSUE MAGNETIC SUSCEPTIBILITY SOURCES

(71) Applicant: Cornell University, Ithaca, NY (US)

(72) Inventors: Qihao Zhang, New York, NY (US); Alexey Dimov, New York, NY (US); Junghun Cho, New York, NY (US); Pascal Spincemaille, New York, NY (US); Yi Wang, New York, NY (US)

(73) Assignee: Cornell University, Ithaca, NY (US)

(21) Appl. No.: 18/858,071

(22) PCT Filed: Apr. 20, 2023
(86) PCT No.: PCT/US23/66015

§ 371 Date: Oct. 18, 2024

Related U.S. Application Data
(60) Provisional application No. 63/332,915, filed on Apr. 20, 2022.

(51) Int. Cl. G01R 33/563; A61B 5/055; G01R 33/56; G06T 15/08

(52) U.S. Cl. CPC G01R 33/56366; A61B 5/055; G01R 33/5601; G01R 33/5608; G06T 15/08; G06T 2210/41

(57) ABSTRACT

Exemplary methods, systems and computer-accessible medium can be provided to generate images of tissue magnetism property from complex magnetic resonance imaging data using the Bayesian inference approach. The tissue magnetic susceptibility sources are organized into multiple components that differentially affect magnetic resonance susceptibility imaging signal, which is utilized to determine these susceptibility components. Exemplary methods, systems and computer accessible medium further enables susceptibility source determination. Thus, according to the exemplary embodiment, system, method and computer-accessible medium can be provided for determining magnetic susceptibility information and other tissue properties associated with at least one structure.

### TECHNICAL FIELD

This invention relates to magnetic resonance imaging, and more particularly to studying tissue magnetic properties using signals collected in magnetic resonance imaging.

### BACKGROUND

Quantitative susceptibility mapping (QSM) in magnetic resonance imaging (MRI) has received increasing clinical and scientific interest. QSM is useful for characterizing and quantifying magnetic chemical compositions such as iron in brain tissue. Tissue composition of these compounds may be altered in various neurological disorders. QSM is able to unravel novel information related to the magnetic susceptibility (or simply referred as susceptibility), a physical property of underlying tissue that depends on magnetic chemical compositions. Due to the prevalence of iron in brain tissue and their active involvement in brain functions, QSM is in general very useful for studying neurological disorders. Therefore, accurate mapping of the magnetic susceptibility induced by iron and other magnetic sources will provide tremendous benefit to clinical researchers to probe the structure and functions of human body, and to clinicians to better diagnose various diseases and provide relevant treatment.

The gradient echo (GRE) sequence is widely accessible and commonly used to acquire data for QSM. Asymmetric spin echo (ASE) sequence can also be used to acquire for QSM. The phase data from GRE and ASE sequences are determined by the inhomogeneity susceptibility field. Both GRE and ASE sequences with their acquired complex image data are referred to as magnetic resonance susceptibility imaging, or simply as complex MRI signal. Image data can be acquired using cartesian sampling with one or many lines in k-space sampled per TR, which is known as standard gradient echo imaging or echo planar imaging for GRE sequence. Image data comprise magnitude information with T2* contrast and phase information. QSM reconstruction can be performed using the approach of estimating the magnetic field from all echo signals acquired various echo times and then performing the field to magnetic susceptibility inversion.

MRI has been used as an essential tool for studying lesions in their body. For example, conventional MRI, including T1 weighted imaging before and post gadolinium injection and T2 weighted imaging with and without fluid attenuated inversion recovery (FLAIR), are used to identify MS lesion size, location, and their dissemination in space and time. However, conventional MRI has difficulty in sensitizing MS disease activities and brain tissue therapeutic responses behind the sealed blood brain barrier, including ongoing chronic active inflammation. QSM has been developed and applied for monitoring chronic active inflammation in MS.

In general, magnetic susceptibility affects the complex MRI signal in a nonlinear manner. Furthermore, biophysical decomposition of susceptibility contributors, including deoxyheme iron in vasculature, myelin, and other diffuse background neural susceptibility in brain tissue, and possible contrast agents transport in vasculature add complexity to the nonlinear signal models. The corresponding inverse problems of nonlinear signal models are nonconvex and their solutions challengingly.

### SUMMARY

Implementations of systems and methods for collecting and processing multiecho (including the single echo as a special case, including acquisition using cartesian, radial, spiral and other trajectories in k-space, including many k-space lines per TR or echo planar imaging) complex MRI signals of a subject, and reconstructing maps of physical properties intrinsic to the subject (e.g., magnetic susceptibility, venous deoxyheme iron, myelin fibers, oxygen extraction) are described below. In some implementations, complex MRI signal data corresponding to a subject can be transformed into susceptibility-based images that quantitatively depict the structure and/or composition and/or function of the subject. Using this quantitative physical property map, one or more images of the subject can be generated and displayed to a user. The user can then use these images for diagnostic or experimental purposes, for example to investigate the structure and/or composition and/or function of the subject, and/or to diagnose and/or treat various conditions or diseases based, at least in part, on the images. As one or more of the described implementations can result in susceptibility-based images having higher quality and/or accuracy compared to other susceptibility mapping techniques, at least some of these implementations can be used to improve a user's understanding of a subject's structure and/or composition and/or function, and can be used to improve the accuracy of any resulting medical diagnoses or experimental analyses.

In general, one aspect of the invention disclosed here enables quantitative study of tissue magnetic susceptibility by obtaining multiecho complex magnetic resonance susceptibility imaging data collected by a magnetic resonance scanner, wherein the multiecho complex magnetic resonance susceptibility imaging data comprises magnitude and phase information or real and imaginary information regarding the object; decomposing the object magnetic susceptibility sources into multiple components differentially affecting the multiecho complex magnetic resonance susceptibility imaging data; determining a distribution of a susceptibility compartment in the object from processing the multiecho complex magnetic resonance susceptibility imaging data, wherein processing involves a deep neural network that is trained on simulated multiecho complex magnetic resonance susceptibility imaging data, wherein the simulation comprises synthesizing susceptibility compartments in a voxel according to an object configuration, synthesizing multiecho complex magnetic resonance susceptibility imaging signal for the object configuration by summing signal contributions from all components in a voxel according to known physical laws, wherein the known physical laws include signal dephasing generated by magnetic susceptibility sources according to the dipole field and generating a voxel value for a susceptibility compartment for the object configuration; generating the one or more images of the object based on the determined distribution of the susceptibility compartment in the object; and presenting, on a display device, the one or more images of the object.

In some implementation, a susceptibility compartment involves deoxyheme iron in vasculature, and one image of the object generated is oxygen extraction fraction map.

In some implementation, a susceptibility compartment is diamagnetic or paramagnetic.

In some implementation, the known physical laws used in simulation include water proton diffusion.

In some implementation, the susceptibility compartments and the signal components within a voxel are represented in a digital twin.

In some implementation, the simulation involves vasculature, wherein vasculature includes capillaries, venules and veins with deoxyheme iron.

In general, another aspect of the invention disclosed here enables a method for generating one or more images of an object, the method comprising: obtaining a multiecho complex magnetic resonance susceptibility imaging data collected by a magnetic resonance scanner, wherein the multiecho complex magnetic resonance susceptibility imaging data comprises magnitude and phase information regarding the object; decomposing the object magnetic susceptibility sources into at least two compartments differentially affecting the multiecho complex magnetic resonance susceptibility imaging data wherein one compartment is paramagnetic; determining a distribution of a susceptibility compartment in the object from processing the multiecho complex magnetic resonance susceptibility imaging data, wherein processing comprises modeling the multiecho complex magnetic resonance susceptibility imaging data through summing signal contributions from all components in a voxel according to known physical laws, wherein the known physical laws include signal dephasing generated by magnetic susceptibility sources according to the dipole field, and performing a spatial deconvolution to extract the susceptibility compartment; generating the one or more images of the object based on the determined distribution of the magnetic source component in the object; and presenting, on a display device, the one or more images of the object.

In some implementation, the compartment being paramagnetic contains deoxyheme iron in vasculature and one image of the object oxygen extraction fraction map.

In some implementation, another compartment is myelin or diffuse paramagnetic iron.

In some implementation, the susceptibility imaging data modeling takes a closed form and processing the multiecho complex magnetic resonance susceptibility imaging data involves an iterative optimization.

In some implementations, processing the multiecho complex magnetic resonance susceptibility imaging data involves a deep neural network.

In general, another aspect of the invention disclosed here enables a method for determining a particle distribution in an object using magnetic resonance imaging, the method comprising obtaining magnetic resonance imaging data from the object, wherein magnetic resonance imaging involves using a contrast agent and includes dynamic phase imaging; determining a particle distribution in the patient from magnetic resonance imaging data, comprising generating a vasculature from magnetic resonance imaging data, and calculating tissue perfusion from dynamic phase imaging; and presenting, on a display device, the determined particle distribution.

In some implementation, generating a vasculature involves a branching algorithm including capillaries as terminal branches and guidance from magnetic resonance imaging data.

In some implementation, the contrast agent is a paramagnetic nanoparticle and magnetic resonance imaging includes pre-injection and equilibrium phase imaging.

In some implementation, calculating tissue perfusion involves fitting the transport equation.

In some implementation, the particle is the microsphere used in transarterial embolization, and the particle distribution is used to calculate the lung shunt fraction.

Some of the implementations described below for the magnetic dipole inversion using incorporation of multiecho complex data based cost function, preconditioning, deep neural network, and enforcement of regional susceptibility contrasts are disclosed to improve susceptibility map quality including suppression of artificial signal variation in regions of low susceptibility contrasts and to provide accurate susceptibility values with precise fidelity to the acquired multiecho complex MRI data, which are pressing concerns to ensure quantification accuracy, experimental repeatability and reproducibility. In some implementations, the object comprises a cortex, a putamen, a globus pallidus, a red nucleus, a substantia nigra, or a subthalamic nucleus of a brain, or a liver in an abdomen, and generating one or more images of the object comprises generating one or more images depicting the cortex, the putamen, the globus pallidus, the red nucleus, the substantia nigra, or the subthalamic nucleus of the brain. In some implementations, the operations further comprise of quantifying, based on the one or more images, a distribution of contrast agents introduced into the object in the course of a contrast enhanced magnetic resonance imaging procedure. The contrast agent passage through tissue is modeled according to a flow velocity and exchange coefficients. The contrast agent passage is captured on many timeframes of time resolved imaging and is used to determined velocity and exchange coefficients by solving the inverse problem of transport equation. This information on contrast agent transport through tissue vasculature is used in vasculature modeling and computational fluid dynamics modeling to predict drug distribution in intravascular delivery.

The details of one or more embodiments are set forth in the accompanying drawings and the description below. Other features and advantages will be apparent from the description and drawings, and from the claims.

### DESCRIPTION OF DRAWINGS

FIG. 1A-1B. Modeling gradient echo signal magnitude decay rates. A) αR₂′ and R₂* for a single slice within an in vivo brain. Both maps have similar appearance with the values laying in the same range. B) Calibration parameter estimation. Relation between R₂′ and R₂* across a set of anatomical ROIs is in good agreement with the disclosed model R₂*=αR₂′, with α≈1.98.

FIG. 2A-2B R₂′ and R₂*-based separation of magnetic sources in vivo. A) R₂′ and R₂*-based separation of magnetic sources. Good overall spatial agreement between χ⁺ and χ⁻ obtained by two techniques can be observed. B) Correlation analysis. Linear regression of the χ⁺ and χ⁻ averaged over 182 lesions from 5 MS patients demonstrated high degree of agreement between two techniques

FIG. 3A-3C. A.) An MS subject's T2FLAIR, MWF and −χ⁻ estimated with R₂* χ⁻ separation. B) Pointwise correlation between MWF and χ_{R}_{₂}_{*}⁻ for the slice in shown in 2A. C) Pointwise correlation between χ_{R}_{₂}_{′}⁻ and χ_{R}_{₂}_{*}⁻

FIG. 4. Network structure of mcQQ-NET. mcQQ-NET consists of 2 4D Unet, one for mGRE phase and the other for mGRE magnitude input. Each Unet consists of an encoding and decoding path with 18 convolutional layers with 3×3×3 kernel (blue), 4 max pooling layers with 2×2×2 kernel (green), 4 deconvolution layers with 2×2×2 kernel (red), 4 feature concatenations (purple), 1 convolutional layer with 1×1×1 kernel (yellow). Linear combination on the Y, v, χₙ channels of output of the 2 Unet (gray) and element-wise Tan h function were applied for parameter boundary setting (cyan).

FIG. 5. Comparison between the OEFs obtained by QQ and mcQQ against truth in simulation. With Large low lesion OEF. The numbers in black indicates mean error. Compared to QQ, mcQQ provides greater accuracy (mean error: 0.39 vs. −0.03%).

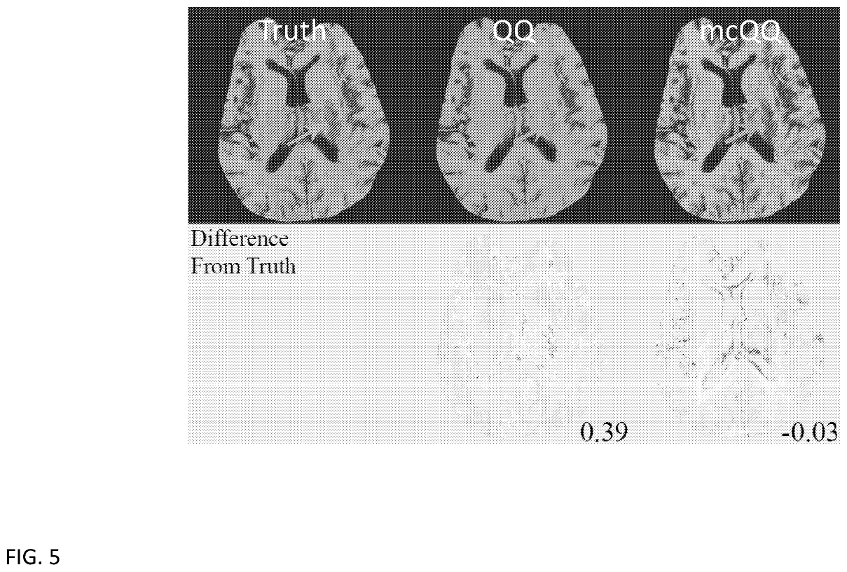

FIG. 6. Vasculature in the brain digital twin. left: large arteries and veins; mid: zoom small arteries, arterioles, venules and small veins; right: capillary density.

FIG. 7 show example processes for mapping tissue magnetic susceptibility.

FIG. 8 is a block diagram of an example computer system.

Like reference symbols in the various drawings indicate like elements.

### DETAILED DESCRIPTION

Implementations of systems and methods for collecting and processing magnetic resonance imaging (MRI) signals of a subject, and reconstructing maps of biophysical properties intrinsic to the subject (e.g., magnetic susceptibility) are described below. The noninvasiveness and rich tissue contrasts of MRI are explored and utilized to characterize pathological, physiological and therapeutical changes in brain tissues for monitoring neurological patients. In some of the disclosed implementations, magnetic susceptibility contrasts are investigated for tissue characterization. In some of the disclosed implementations, quantitative susceptibility mapping (QSM) and related postprocessing of magnetic resonance susceptibility imaging data are developed and applied for studying tissue magnetic properties. Though both gradient echo and asymmetric spin echo can be used to acquire multiecho magnetic resonance susceptibility imaging data, we refer to both types of sequences in the following as multiecho gradient echo (mGRE).

Implementations of systems and methods for collecting and processing MRI signals of a subject, and reconstructing maps of physical properties intrinsic to the subject (e.g., magnetic susceptibility) are described below. Some of the disclosed implementation enable study of separate components in tissue magnetic susceptibility sources. For brain tissue, there are three major components, paramagnetic deoxyheme iron in vasculature including capillaries, venules and veins, diamagnetic myelin fibers in white matter, as well as in gray matter to a lesser degree, and diffuse background neural susceptibility, which includes paramagnetic ferritin iron in neurons and other glia cells. These three components affect mGRE signal differentially. Diamagnetic myelin contributes negatively to net susceptibility in a voxel, while paramagnetic deoxyheme iron in vasculature contributes positively to net susceptibility in a voxel. The net susceptibility in a voxel can be determined from mGRE phase data according to dipole deconvolution, which is quantitative susceptibility mapping (QSM). Both myelin and deoxyheme are in cylindrical spatial distribution and affect mGRE magnitude signal differently from diffuse background neural susceptibility. These differential effects on mGRE signal by the three susceptibility source components are made use for separating the three components in this disclosed invention.

Some of the disclosed implementations enable the positive and negative susceptibility sources separation based on mGRE data alone. This eliminates the practice hurdle in prior arts that require acquisition of another spin echo sequence, adding scan time and misregistration error. Multiecho gradient echo data alone allows separate determination of dia- and para-magnetic sources, which is established using quantitative susceptibility mapping of phase and a R2* model of magnitude, and has validated using quantitative comparison with histology. For determination of dia- and para-magnetic sources, QSM processing of phase is combined with a R2* model of magnitude of mGRE data. This combined problem is solved iteratively using iterative optimization, such as Gauss-Newton iterations. This mGRE data alone for positive and negative susceptibility separation is advantageous over a prior R2′ method that requires additional T2 mapping, Magnetic source separation was performed in in vivo (17 multiple sclerosis (MS) patients) and ex vivo (a whole brain and 3 MS brain tissue blocks). Both R2* and R2′ methods were found to be well correlated in vivo data. R2* based positive and negative susceptibility sources estimation in ex vivo MS lesions correlated with optical density of lesion histology.

Some of the disclosed implementations enable accurate oxygen extraction fraction (OEF) mapping from mGRE data alone without vascular challenge. The mGRE magnitude data is modeled according to quantitative blood oxygen level dependent (qBOLD), which is combined with QSM processing of mGRE phase data, forming qBOLD+QSM=QQ. A multiecho complex QQ (mcQQ) method to map oxygen extraction fraction (OEF) is developed by modeling the data in the complex domain of precise gaussian noise, instead of the domain of susceptibility and signal magnitudes used in the prior art. mcQQ enhances detection of lesion OEF abnormality in stroke. Quantitative mapping of oxygen extraction fraction (OEF) is critical to evaluate brain tissue viability and function in neurologic disorders. An integrated model of mcQQ in the mGRE complex data domain has been developed to map OEF from a routine gradient echo MRI without the need for vascular challenges, and mcQQ inversion can be obtained using deep learning or a deep neural network. mcQQ improves fidelity to data noise characteristics using complex domain. The disclosed mcQQ provided more accurate OEF in simulation and an improved sensitivity to OEF abnormalities in ischemic stroke patients, compared to the current QQ method. Accurate QSM improve mapping of tissue oxygen extraction fraction, as well as other applications such as fat or myelin water fraction mapping. Various aspects of our invention are exemplified in the specific embodiments in the following implementations of systems and methods for collecting and processing MRI signals of a subject, and reconstructing maps of physical properties intrinsic to the subject. QSM corrects errors in omitting background neural tissue magnetic susceptibility in prior methods by combining qBOLD modeling of gradient echo (GRE) magnitude data and QSM processing of GRE phase data (QQ-qBOLD+QSM). The fundamental physics of deoxyheme iron effects on GRE signal, including fMRI and quantitative blood oxygenation level (Y) dependent (qBOLD) models, is the dephasing or off-resonance,

$$δ ω = δ χ \frac{{ω}_{0}}{2} {( \frac{a}{ρ} )}^{2}$$

cos 2φ sin² θ caused by a long solid cylinder of radius α and δχ susceptibility relative to background at a distance ρ with azimuthal/polar angle φ/θ (in SI unit). Prior methods for OEF (σ=1−Y) including qBOLD assume background neural susceptibility χₙ=arterial blood susceptibility χ_{ba} (≈−108.3 ppb).

δχ_{qBOLD}=ψ_{Hb}Δχ_{Hb}·σ

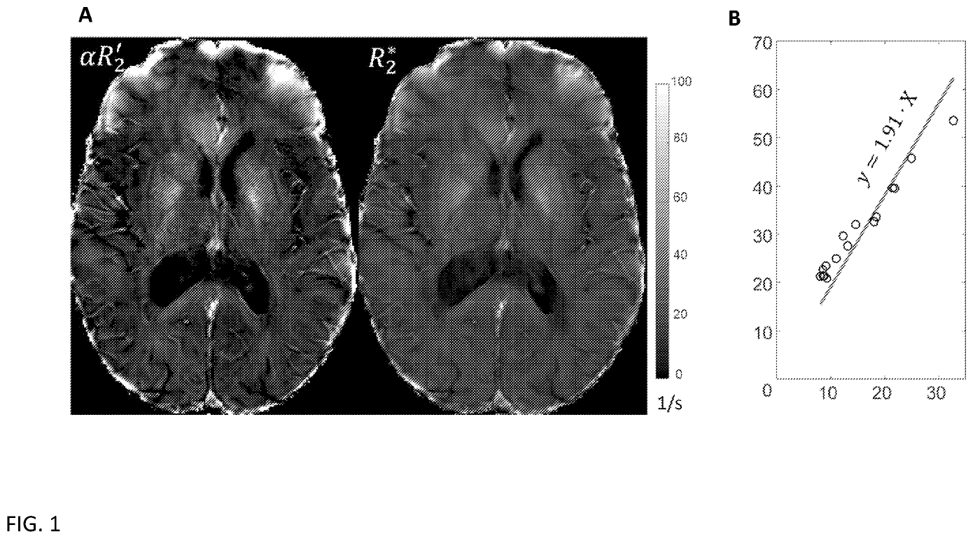

However, background neural χₙ differs heterogeneously from arterial blood χ_{ba}, particularly in deep gray matter with paramagnetic neural iron in ferritin (100 ppb) and white matter with diamagnetic myelin tracts (−50 ppb); the background neural χₙ dominated by diffuse ferritin iron varies with age and sex. These substantial variations in space and dependences on age and sex cause erroneous bias (FIG. 1). Background χₙ correction is needed and is enabled in QQ using QSM processing of the already acquired phase data without adding scan time,

$${δχ}_{QQ} = {ψ}_{Hb} {Δχ}_{H b} \cdot + {χ}_{b a} - {χ}_{n}$$

In the above Equations, ω₀ is the resonance frequency, 128 MHz @3T, ψ_{Hb}≡0.09 hemoglobin volume fraction at tissue hematocrit Hct=0.357, and Δχ_{Hb}=12522 ppb volume susceptibility difference of deoxy- and oxy-hemoglobin. This QQ innovation substantially improves OEF accuracy. Some implementations of this invention contains detailed improvements on modeling of biophysics processes underlying MRI signal. One specific novel element is brain tissue digital twin with microvasculature, myelin and diffuse neural susceptibility and with blood flow, oxygen extraction and diffusion for modeling precisely mGRE signal, which allows accurate estimation of underlying biophysics parameters.

Some of the disclosed implementations enable a digital twin for accurate signal modeling of mGRE data. The prior art of using a closed form expression for mGRE signal become inaccurate for real tissue with complex compositions. We overcome this problem by creating an in silico form or digital twin for tissue. The digital twin allows a detailed geometric model of tissue components in a voxel, such as magnetic sources, and dynamic signal model, such as spin diffusion in addition to static dephasing. The digital twin represents realistic vasculature of arteries, arterioles, capillaries, venules, and veins according to available angiographic information specific to a test subject and according to biological prior information regarding vasculature. The digital twin represents realistic tissue composition of myelin and diffuse background neural susceptibility source according to QSM and myelin water fraction information specific to a test subject. The digital twin is recalled to synthesize mGRE signal, and general MRI signal, at micron resolution for a voxel, enabling an accurate virtual MRI simulator. Various physiological properties, such as tissue transport of oxygen and contrast agents, can be included in the digital twin to reflect realist MRI signal. This digital twin can be used to develop MRI acquisition, reconstruction and image processing. For studying tissue susceptibility sources, the digital twin provides an in silico truth for validation and a rich source of data to train deep neural network.

Some of the disclosed implementations overcome limitations in quantitative susceptibility mapping (QSM), where the computation robustness and accuracy and the Bayesian prior information affect QSM image quality and practical usage. In some implementations, to reconstruct maps of a subject's magnetic susceptibility, the computation process of finding the susceptibility distribution that optimally fit available mGRE data and tissue structure prior information is made accurate by fitting data at each echo time without compromising the precision in modeling data noise. In some implementation, the computation process of finding the magnetic field from mGRE data is also made accurate by fitting data at each echo time without compromising the precision in modeling data noise. In some implementation, a scaling factor is used to ensure robust initialization of the nonconvex optimization needed to deal with precise signal noise modeling. In some implementation, additional information of regions with low susceptibility contrasts that can be automatically segmented from R2* map is used to improve QSM reconstruction. In some implementation, deep neural network is used for magnetic field estimation and QSM reconstruction. Implementations of these improvements have made QSM technology robust and accurate. For multiecho gradient echo (mGRE) magnetic resonance imaging data obtained by a magnetic resonance scanner, the mGRE data at each echo time is complex, its magnitude and phase information or real and imaginary information regarding the object is well characterized to have Gaussian noise in the domain of complex signal. This mGRE data should be generally interpreted as multiple echoes with both magnitude and phase evolving with echo times. Gradient echo, spin echo and hybrid echo and other type of pulse sequences can be used to acquire these multiple echoes. Therefore, we refer to these data as multiecho complex (MC) data. QSM prior arts typically consist of 1) estimating the magnetic field from multiecho complex signals at all echo times and 2) performing the inversion from magnetic field to magnetic susceptibility. The first step here typically focuses on signal phase, ignoring noise effects on signal magnitude; the second step here invariably has difficulties with the very complex noise property of the estimated field. Consequently, both steps contribute errors to QSM reconstruction. To avoid these two steps and their associated errors, some implementations of our invention improve upon QSM prior arts through estimating a magnetic susceptibility distribution of the object based on the multiecho complex data at each echo time. We construct a cost function relating a data fidelity term associated with a likelihood function of the Gaussian noise in the multiecho complex data at each echo time. The likelihood function for the multiecho complex data at an echo time is Gaussian with the variance same for all echoes but the mean dependent on individual signals, and the product of likelihood functions of all echoes form the resultant likelihood function. The negative log of the likelihood function is the cost function referred to as data fidelity term, and the maximal likelihood estimation corresponds to the minimal cost function. This leads to that the cost function for the QSM reconstruction is a sum of cost functions of all individual echoes. Extending to the Bayesian inference framework, the prior probability is the exponential function of the negative regularization term, the prior probability times the likelihood function determines the posterior probability, and the maximum a posterior probability (known as MAP) estimation leads to minimum of a cost function consisting of data fidelity term from likelihood and regularization term from prior probability. The data fidelity term involves summing a comparison between the measured signal and modeled signal at each echo. This comparison can be a difference expressed in an Lp norm (p≥0, for example, p=2 for Gaussian noise model). The modeled signal involves temporal evolution in both magnitude and phase. As the field to susceptibility inverse problem is ill-posed, additional information is needed for QSM reconstruction, which is a regularization term associated with a structure prior information. Then the magnetic susceptibility distribution is determined based on minimizing the cost function. We extend the approach of going back to individual echo data to addressing errors in the first and second steps of QSM prior arts. Therefore, the first step is improved by calculating a magnetic field experienced by the tissue in the object based on modeling the multiecho complex data with consideration of noise in both magnitude and phase information at each echo time, which would allow precise modeling noise in both magnitude and phase data. The second step is improved by replacing the temporal phase evolution with echo time in the multiecho complex data with a phase factor determined by the calculated magnetic field, which would allow proper estimation of noise weighting in formulating the data fidelity term. This is a nonlinear nonconvex optimization problem, which depends on initialization or may be trapped in local minima. We devise a scaling method for solving nonlinear nonconvex optimization problem without concerns in initialization trapping. We scale the magnetic field down to be very small, which alleviates initialization problems and allows linearization of phase factors. The magnetic field is divided first by a factor for estimating the magnetic susceptibility. The small susceptibility values can be reliably searched. Then, the output magnetic susceptibility is multiplied by the factor, to obtain the properly values susceptibility distribution of the object. For tissue with fat, we solve the fat water separation problem for obtaining the magnetic field in the presence of phase and magnitude effects from chemical shifts of fat and other compounds. This fat-water separation problem is a nonlinear nonconvex optimization problem that is dependent on initialization. We device a deep neural network approach with or without training (supervised or unsupervised training) to overcome this initialization problem. The magnetic field, the water fraction and the fat fraction in a voxel can be computed using a deep neural network. We further improve the use of structure prior information for effective suppression of artifacts in QSM reconstruction. The concept of penalizing susceptibility variation in a region of uniform susceptibility or zero susceptibility contrast can be extended to include regions of small susceptibility contrasts at graded penalties. A region with a susceptibility contrast slightly larger than zero can be characterized by a slightly lower penalty in the cost function. In this manner, estimating the magnetic susceptibility distribution include steps of identifying a region of interest in a tissue that has a low susceptibility contrast according to a characteristic feature and adding a second regularization term associated with enforcing the known low susceptibility contrast in the region of interest in the tissue. We use R2* values as the characteristic feature for identifying regions at various susceptibility contrasts. R2* values can be readily obtained from multiecho complex data. The region of interest at a given susceptibility contrast can be determined by similar R2* properties of contiguous voxels, and the penalty there depends on a representative R2* value in the region of interest. We have developed several solver methods to perform the cost function minimization. One solver involves preconditioning that uses small search steps in low susceptibility regions and large search steps in high susceptibility regions (absolute value of susceptibility and water is zero-reference). Another solver for estimating the magnetic susceptibility distribution is realized using a deep neural network (DNN). The DNN can be trained under supervision of labeled data, including data generated from known biophysics models. The DNN can be trained with unlabeled data according to biophysics model of signal. The DNN can also be used without training to process a test data by minimizing a cost function based on the biophysics model of signal.

In general, another implementation for generating one or more images of an object comprises obtaining multiecho complex magnetic resonance imaging data collected by a magnetic resonance scanner, wherein the multiecho data is complex and comprises magnitude and phase information regarding the object, calculating a magnetic susceptibility distribution using quantitative susceptibility mapping processing of the obtained multiecho complex magnetic resonance imaging data, determining oxygen extraction fraction distribution of the object based on the multiecho complex data, comprising determining a cost function, the cost function involving a comparison between a magnitude image of obtained multiecho complex magnetic resonance imaging data and a magnitude image of a modeled complex signal with a consideration of deoxyheme-iron effects at each echo time, and a comparison between the calculated susceptibility and a susceptibility decomposition model including a deoxyheme-iron component, and a regularization term on oxygen extraction fraction and venous blood volume, and minimizing the cost function, and presenting, on a display device, one or more images of the object generated based on the determined oxygen extraction fraction distribution. The regularization term may be explicitly expressed such as a sparsity form or implicitly expressed such as learned by a deep neural network.

In general, another implementation for generating one or more images of an object comprises obtaining multiecho complex magnetic resonance imaging data collected by a magnetic resonance scanner, wherein the multiecho data is complex and comprises magnitude and phase information regarding the object, determining myelin distribution of the object based on the multiecho complex data, comprising determining a cost function, the cost function including a data fidelity term involving a comparison between the obtained multiecho complex magnetic resonance imaging data and a modeled complex signal at each echo time with a consideration of myelin effects on signal magnitude and phase, and a regularization term, and minimizing the cost function, and presenting, on a display device, one or more images of the object generated based on the determined oxygen extraction fraction distribution. The regularization term may be explicitly expressed such as a sparsity form or implicitly expressed such as learned by a deep neural network.

As examples of specific implementations, the consideration of deoxyheme-iron effects on signal magnitude and phase may include a model of deoxyheme-iron distributed in cylinders and nonblood susceptibility sources distributed diffusely in space for determining the modeled complex signal; the comparison between the obtained multiecho complex magnetic resonance data and the modeled complex signal may comprise an Lp norm of the difference of the obtained multiecho complex magnetic resonance data and the modeled complex signal, and/or the more images of the object include the nonblood susceptibility distribution and/or venous blood volume; the regularization term may include a sparsity of edges in oxygenation and/or venous blood volume; minimizing the cost function may include a dipole deconvolution step and the regularization includes a sparsity of edges in tissue susceptibility and/or penalties on variations of susceptibility values in regions; the regularization term may include a sparsity of temporal evolution along echo time of the magnitude of the modeled complex signal; minimizing the cost function may include coordinate descent along axes defined by variables of oxygenation and venous blood volume; the regularization term may be implicitly formed and determining the oxygen extraction fraction distribution is realized using a deep neural network, wherein the deep neural network may be trained with labeled data or unlabeled data, or is untrained, and/or the deep neural network is trained with network weights updated with a test data.

The modeled complex signal with a consideration of deoxyheme-iron effects on signal magnitude and phase at each echo time include modeling deoxyheme-iron as cylinders and other susceptibility sources as diffuse and calculating corresponding effects on signal magnitude and phase at each echo time. Deep neural networks can be used to minimize the cost function.

In some implementations, to reconstruct maps of a subject's magnetic susceptibility, the computation process of finding the susceptibility distribution that optimally fit available susceptibility imaging data, such as gradient echo MRI (GRE) data, and tissue structure prior information is made accurate by fitting data at each echo time without compromising the precision in modeling data noise. For GRE magnetic resonance imaging data obtained by a magnetic resonance scanner, the GRE data at each echo time is complex, its magnitude and phase information or real and imaginary information regarding the object is well characterized to have Gaussian noise in the domain of complex signal. This GRE data should be generally interpreted as multiple echoes with both magnitude and phase evolving with echo times. Gradient echo, spin echo and hybrid echo and other type of pulse sequences can be used to acquire these multiple echoes.

In some implementations, reconstructing maps of a subject's magnetic susceptibility comprises using the magnitude and phase information and additional information. The additional information may comprise known tissue structural information, such as sparsity in certain mathematical representations, including various norms of edge operations, total variation, and wavelets. The additional information may be learned from training dataset using deep neural network.

In some implementation, determining the quantitative susceptibility map comprises using a data fidelity term associated with a likelihood function associated with a Gaussian noise. The Gaussian likelihood function may be imposed rigorously in the complex gradient echo data domain, or approximately in various data domain, such as the phase or field data domain.

In some implementations, to characterize a MS lesion, the susceptibility distribution of the lesion is evaluated and compared with the susceptibility distribution of surrounding normal appearing tissue. For example, the region of tissue surrounding in the T2 region of the lesion may comprise a layer of normal appearing white matter and/or gray matter surrounding the lesion. Determining characteristic features comprises using a susceptibility value measured on the quantitative susceptibility map.

In general, an example embodiment is a method for generating one or more images of an object, the method comprising obtaining complex magnetic resonance data collected by a magnetic resonance scanner, wherein the data is complex and comprises magnitude and phase information regarding the object, estimating a magnetic susceptibility distribution of the object based on the obtained complex magnetic resonance data, wherein estimating the magnetic susceptibility distribution of the subject comprises determining a cost function, the cost function including a data fidelity term involving modeling a magnetic susceptibility effect on complex magnetic resonance data, a regularization term involving a first region with a low feature of susceptibility contrasts, and a second region with a feature of susceptibility contrasts different from the feature of susceptibility contrasts of the first region, minimizing the cost function, determining a magnetic susceptibility distribution based on the act of minimizing the cost function, and presenting, on a display device, one or more images of the object generated based on the determined magnetic susceptibility distribution.

1. Susceptibility Source Separation from Gradient Echo Data Alone by Incorporating Magnitude Decay Approximation into Quantitative Susceptibility Mapping Phase Processing

In this section, we demonstrate feasibility of separating magnetic sources in quantitative susceptibility mapping (QSM) by incorporating magnitude decay rates R₂* in gradient echo (GRE) MRI. Magnetic susceptibility source separation was developed using R₂* and compared with a prior method using R₂′=R₂*−R₂ that required an additional sequence to measured the transverse relaxation rate R₂. Both susceptibility separation methods were compared in multiple sclerosis (MS) patients (n=17). Susceptibility values of negative sources estimated with R₂*-based source separation in a set of enhancing MS lesions (n=44) were correlated against longitudinal myelin water (MWF) fraction changes. In in vivo data, linear regression of the estimated χ⁺ and χ⁻ susceptibility values between the R₂* and the R₂′ based separation methods performed across 182 segmented lesions, revealed correlation coefficient r=0.96 and slope close 0.99. Correlation analysis in enhancing lesions revealed a significant positive association between the χ⁻ increase at 1-year post-onset relative to 0y and the MWF increase 1y relative to 0y (β=−0.144, 95% CI: [−0.199, −0.1], p=0.0008) and good agreement between R₂′ and R₂* methods (r=0.79, slope 0.95). Separation of magnetic sources based solely on gradient echo (GRE) complex data is feasible by combining magnitude decay rate modeling and phase based QSM and χ⁻ change may serve as a biomarker for myelin recovery or damage in acute MS lesions.

### 1.1. INTRODUCTION

Multiple sclerosis (MS) is a debilitating autoimmune disease in the central nervous system characterized by the presence of demyelinated lesions. Ongoing chronic inflammation can be detected by the presence of increased iron at the rims of MS lesions using quantitative susceptibility mapping (QSM) phase processing of gradient echo (GRE) MRI data. Progression of demyelination can be monitored using noninvasive MRI methods such as myelin water fraction mapping, which requires acquisition with a spin echo (SE) type pulse sequence. Both iron increase and myelin loss can contribute to the apparent increase of magnetic susceptibility within a lesion QSM. It is desirable to separately quantify myelin and iron from GRE MRI alone for in vivo monitoring of MS disease progression.

Iron and myelin are quantified together in QSM on the basis of their non-zero magnetic susceptibility with respect to water. Additional information is needed to separate diamagnetic myelin and paramagnetic iron when they are present within the same voxel. GRE magnitude decay rate R₂* with various assumptions has been proposed, but its use in MS brains has not been validated. Longitudinal rate R₁, and reversible dephasing rate R₂′=R₂*−R₂ have been also proposed to separate susceptibility sources on QSM, but these approaches require an additional SE acquisition for R₁ and R₂ mapping.

In this invention, we have developed an R₂* based separation of positive and negative susceptibility sources in QSM, a susceptibility source model of two compartments, using solely GRE data. We take advantage of the fact that the susceptibility sources are fairly dilute with small concentrations in tissue with corresponding R₁, R₂, R₂′ and R₂*, approximately proportional to the magnitude of susceptibility source concentration. We compare the R₂* separation with the prior R₂′ separation that was previously used in MS patients.

### 1.2 MATERIALS AND METHODS

### Signal Model and Solver

The dephasing effects of magnetic susceptibility can be modeled according to known physical law as R₂′(r)=r⁺|χ⁺(r)|+r⁻|χ⁻(r)|, Here χ⁺(r) and χ⁻(r) are sought volumetric susceptibilities of positive and negative sources, r⁺ and r⁻ are their corresponding relaxometry constants and are assumed to be spatially invariant and known a priori. At a given voxel, R₂′ can be readily obtained from R₂* and R₂, R₂*(r)=R₂′(r)+R₂(r). Determination of the R₂ map requires acquisition of additional sequences such as multi-echo spin echo, which may not be practical in the clinic due to time constraints, and are not available in existing clinical MRI for retrospective analysis. In the approximation of small concentration, R₂ is proportional to the magnitude of susceptibility sources in both inner sphere effects and outer sphere effects. Correspondingly, the R₂′ linear proportionality can be extended to R₂, and the recent susceptibility source separation work based on R₂′ can be extended to processing gradient echo data alone using a first-order approximation, R₂*(r)≈αR₂′(r), where a is a calibration parameter estimated through the linear fit with zero-intercept within a set of ROIs when both R₂* and R₂′ maps were available (see details in the further sections). With this simplifying assumption, the complex gradient echo signal exponent can be written as:

$${R}_{2}^{*} ( r ) + i 2 π Δ f ( r ) = α {+}^{} \vert {χ}^{+} ( r ) \vert + α {-}^{} \vert {χ}^{-} ( r ) \vert + i γ \cdot d * ( {χ}^{+} ( r ) + {χ}^{-} ( r ) ) \qquad [ 1 ]$$

Here d is the dipole kernel. Eq. 1 requires only gradient echo data as input, and its inverse problem can be formulated as a minimization problem and solved iteratively using a conjugate gradient descent algorithm with Gauss-Newton iterations. In the present work, the following formulation was implemented:

$$\begin{matrix}{‖ {w}_{1} ( r - ( {+}^{} \vert {χ}^{+} \vert + {-}^{} \vert {χ}^{-} \vert ) ) ‖}_{2}^{2} + {‖ {w}_{2} ( f - d * ( {χ}^{+} + {χ}^{-} ) ) ‖}_{2}^{2} \\ \begin{matrix}{χ}^{+ *} , {χ}^{- *} = {arg min}_{{χ}^{+} , {χ}^{-}} & + 2 {λ}_{1} {\vert {M}_{mag} ∇ ( {χ}^{+} + {χ}^{-} ) \vert}_{1} + {λ}_{1} {\vert {M}_{r} ∇ {χ}^{+} \vert}_{1} + {λ}_{1} {\vert {M}_{r} ∇ {χ}^{-} \vert}_{1}\end{matrix} \\ + {λ}_{2} {‖ {M}_{CSF} ( {χ}^{+} - \overline{{χ}_{CSF}^{+}} ) ‖}_{2}^{2} + {λ}_{2} {‖ {M}_{CSF} ( {χ}^{-} - \overline{{χ}_{CSF}^{-}} ) ‖}_{2}^{2}\end{matrix} \qquad [ 2 ]$$

Here r is R₂′ or R₂*, r⁺ and r⁻ are properly scaled relaxometric constants (137 Hz/ppm for R₂′ and α·137 Hz/ppm for R₂*), f is the local field, λᵢ are regularization parameters, ∇ is a gradient operator, M_{mag} is a binary edge mask derived from the magnitude image, Mᵣ is a binary edge mask derived from r similar to M_{mag}, M_{CSF} is a binary mask of CSF or, in case of a phantom, pure water and

$$\overline{{χ}_{CSF}^{\cdot }}$$

are χ⁺ and χ⁻ averaged over M_{CSF}. The data weight w₂ reflects the reliability of the estimated frequency of each voxel, while w₁ reduces the effects of unreliable r estimations. Susceptibility values violating physical constraints (χ⁺>0 and χ⁻<0) were reset to zero.

### In Vivo Imaging Experiments

The described R₂* separation of positive and negative susceptibility sources was developed and compared with the prior R₂′ separation method on data from multiple sclerosis (MS) patients (n=17), and this study was performed under an IRB approved retrospective protocol. Subjects were scanned on a 3T clinical MR scanner (Siemens Healthineers, Erlangen, Germany). A 3D multi-echo gradient echo (GRE) sequence was acquired with the following parameters: voxel size=0.75×0.75×3 mm³, first TE=6.3 msec, ΔTE=4.1 msec, TR=48 msec, flip angle=15 degrees, readout band width (rBW)=260 Hz/pixel. FAST-T2 was used for R2 mapping. Imaging parameters were: 3D spiral acquisition, TR/TE=7.5/0.5 ms, sequence TR=2000 ms, T2prep echo times=0 (T2prep pulse turned off), 7.5, 17.5, 67.5, 147.5, and 307.5 ms, flip angle=10°, number of spiral leaves per stack=32, number of signal averages=1, matrix size=192×192×32 interpolated to 256×256×32, voxel size=0.9×0.9×5 mm³. For lesion identification and tracing, the following sequences were acquired: T2 FLAIR (voxel size=1×1×1 mm³, TE=448 ms, TR=7600 ms, TI=2450 ms, rBW=780 Hz/pixel), T2 fast spin echo (FSE) (voxel size=0.5×0.5×3 mm³, TE=93 ms, TR=5840 ms, flip angle=140°, rBW=225 Hz/pixel) and T1w inversion prepared fast spoiled gradient echo (voxel size=1×1×3 mm³, TE=2.3 ms, TR=2300 ms, TI=900, flip angle=8°, rBW=200 Hz/pixel).

In 12 MS patients (mean age/disease-duration 40 y/7.6 y), there were 44 new Gadolinium-enhancing lesions that were followed up 1y later, and their MRI included FAST-T2 based MWF mapping. We investigated early χ⁻ change and 1st-y myelin recovery. χ⁻ (myelin susceptibility) was estimated using both prior R₂′ and described R₂* approaches. Linear mixed effects model with a random effect for patient and with adjustment for lesion volume was used to study association between χ⁻ and MWF.

Here and elsewhere, R₂* and R₂ were estimated using Auto-Regression on Linear Operations (ARLO). To estimate the frequency maps, the multi-echo phase data were fitted to a nonlinear model. Then, the result was spatially unwrapped, after which background field was removed using PDF. QSM was reconstructed using Morphology Enabled Dipole Inversion (MEDI). All co-registrations were performed using the FSL FLIRT algorithm.

Lesion tracing was done manually by an experienced neuroradiologist on the T2 FLAIR images, and prepared segmentations were registered onto GRE images. Lesion-wise means of χ⁺ and χ⁻ estimated using both r=R₂′ and r=R₂^{*} in Eq.2 were recorded. Estimation of the calibration parameter α was performed in a single in vivo case on a set of ROIs placed within right and left globus pallidus, putamen, caudate nuclei, thalamus, red nuclei, subthalamic nuclei, posterior white matter tracks splenium and rostrum of corpus callosum.

### 1.3 RESULTS

FIG. 1 shows the relational between R₂′, R₂* exemplified in a single MS patient. The linear regression resulted in α=1.91 (r²=0.94, p<0.05).

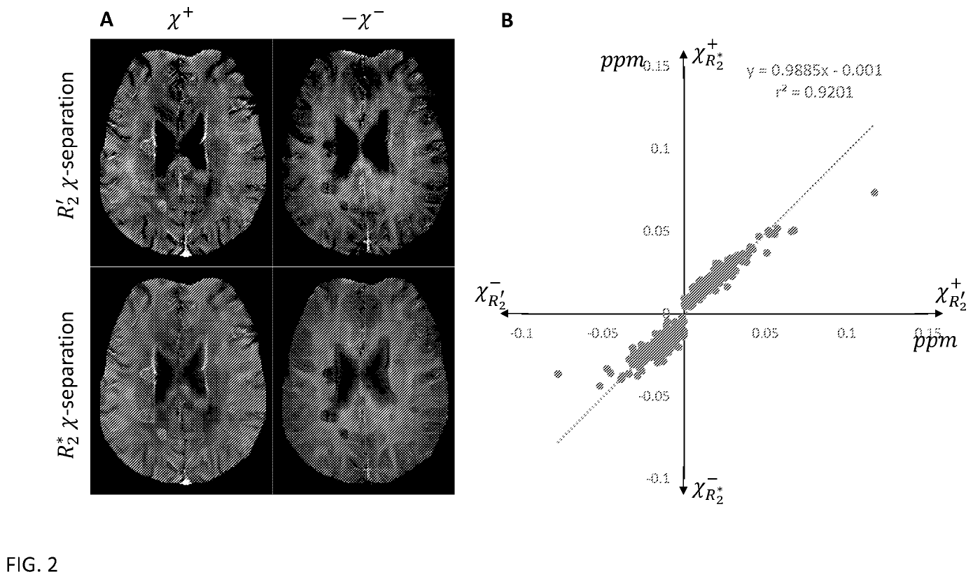

FIG. 2 shows representative results of magnetic source separations performed by both R₂* and R₂′ methods. FIG. 2A shows the T2w, T2FLAIR and T1W images. Good agreement in the generated contrast between the lesion and surrounding NAWM as well as distribution of sources within the lesions can be observed in both χ⁺ and χ⁻ maps in FIG. 2B. Overall, 182 segmented lesions from the 5 patients were obtained. Linear regression (FIG. 2C) of the estimated χ⁺ and χ⁻ susceptibility values between the R₂* and the R₂′ based separation methods demonstrated good agreement, with correlation coefficient r=0.96 and slope close 0.99. Results of the Bland-Altman analysis of the lesion components' susceptibilities (FIG. 2D) shows good agreement of both techniques

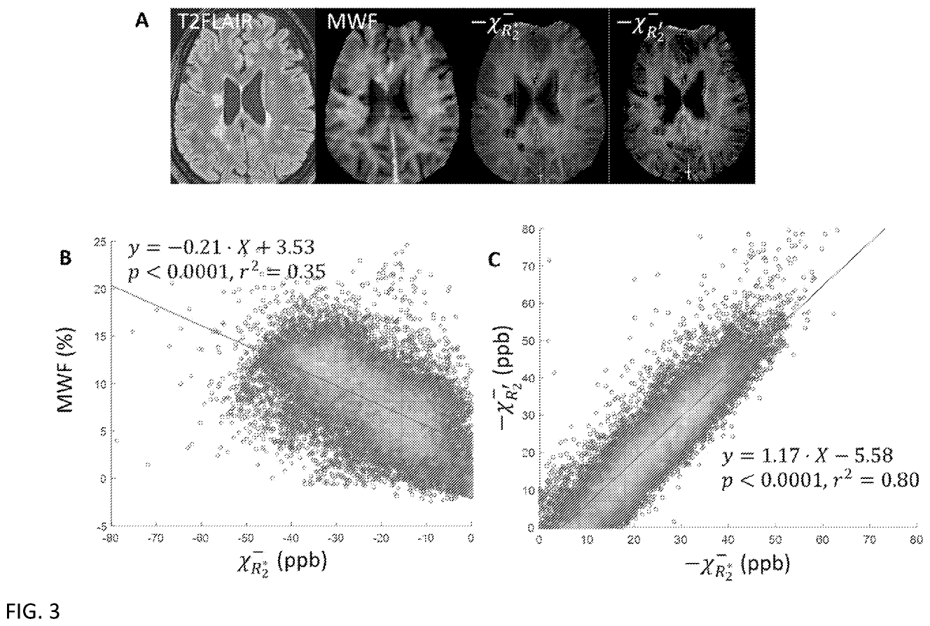

FIG. 3A exemplifies a co-registered MWF and −χ⁻ reconstructions in one typical MS patient. Overall good spatial agreement was confirmed through correlation analysis (FIG. 3B), with good agreement observed between R₂′ and described R₂* reconstructions (FIG. 3C). A significant positive correlation was found between the χ⁻ increase @ 1y relative to 0y and the MWF increase @ 1y relative to 0y (β=−0.144, 95% CI: [−0.199, −0.1], p=0.0008). Both susceptibility estimations are in a good agreement, with slope of 0.95 and correlation coefficient r=0.79.

### 1.4 FURTHER DESCRIPTION AND CONCLUSIONS

Our in vivo data demonstrates the feasibility of combining gradient echo magnitude decay rate R₂* and phase based QSM measurements for separating negative and positive susceptibility sources in MS. In vivo results show good agreement between the R₂*-based method and a prior R₂′-based method that required additional data acquisition. Comparison of negative susceptibility and myelin water fraction indicates that χ⁻ change estimated by R₂*-based separation may serve as a biomarker for myelin recovery/damage in acute MS lesions.

In the present work, we explored a simplified model that allows for assessment of tissue composition from the gradient echo data alone. The main assumption in our framework is that susceptibility-induced R₂* and R₂* effects are approximately proportional. This can be viewed as a reasonable first order approximation, since both R₂ and R₂′ are linearly proportional to the magnitude of concentrations of susceptibility sources. This assumption is further validated by previously reported direct in vivo measurements of R₂* and R₂′. These data suggest approximation of R₂*≈αR₂′ holds well across the brain, with a for white and grey matter typically varying within the interval [1.5 . . . 2.5], similar to the values of the calibration parameter in the present study. Combined with the reported insensitivity of the results of source separation to scaling of r⁺ and r⁻ within the diapason of ±25% around their nominal values, this relation between relaxation rates explains good agreement achieved between R₂′- and R₂*-based techniques. Although R₂ might have a constant component presently unaccounted in the Eq. 3 and describing diffusion-related effects, this term is expected to have smaller amplitude compared to R₂′. This, combined with modeling of local field in Eq. 5, results in successful R₂*-based source separation.

It should be noted that the R₂* and R₂′ modeling in this work ignores white matter anisotropy. Myelinated axons in the brain are organized in bundles. Their orientation with respect to the main magnetic field differs in various parts of the brain and cannot be estimated without diffusion tensor imaging. This presents a limitation of the current and prior methods of susceptibility source separation, and its influence on the estimated myelin values should be further investigated. Specifically, comparison of our myelin maps with those obtained in previous independent studies reveals noticeable underestimation of myelin content within the globus pallidus. Further development of the method should incorporate effects of sample orientation, source geometry and effects of diffusion. Nevertheless, our data suggest that our approximation produces a good qualitative agreement with independent measurements of iron and myelin within MS lesions. Since R₂* values are also affected by strong field inhomogeneity, R₂* correction is expected to improve overall quality of reconstructed sources maps, especially in the infratentorial brain and above nasal sinuses.

In conclusion, separation of magnetic sources based solely on GRE complex data is feasible by combining R₂* magnitude decay rate modeling and phase. Our work extends the applicability of previously proposed MR quantification of para- and diamagnetic sources to the GRE data alone, which simplifies acquisition protocols and allows retrospective analysis of already existing data.

2. Multi-Echo Complex QSM+qBOLD (mcQQ) for Oxygen Extraction Fraction (OEF) Mapping

In this section, we present a multi-echo complex QQ (mcQQ) method to map oxygen extraction fraction (OEF) is developed by modeling the data in the complex domain of gaussian noise, instead of previous domain of susceptibility and signal magnitudes. mcQQ enhances detection of lesion OEF abnormality in stroke. Quantitative mapping of oxygen extraction fraction (OEF) is critical to evaluate brain tissue viability and function in neurologic disorders. An integrated model of QSM and qBOLD (QSM+qBOLD or QQ) has been developed to map OEF from a routine gradient echo MRI without the need for vascular challenges, and QQ inversion can be obtained using deep learning. In this invention, we describe a multi-echo complex QQ (mcQQ) that improves fidelity to data noise characteristics using complex domain. The described mcQQ provided more accurate OEF in simulation and an improved sensitivity to OEF abnormalities in ischemic stroke patients, compared to the current QQ method.

### 2.1 INTRODUCTION

Quantitative mapping of oxygen extraction fraction (OEF) is critical to evaluate brain tissue viability and function in stroke. An integrated model of quantitative susceptibility mapping and quantitative blood oxygen level dependent magnitude (QSM+qBOLD or QQ) has been developed to consider the OEF effect on both magnitude and phase of a widely available multi-echo gradient echo (mGRE) data, and validated against calibrated fMRI and ¹⁵O-PET. With removing clinically impractical vascular challenges, its clinical feasibility has already been shown in ischemic stroke, multiple sclerosis, and brain cancer. Recently, deep learning allowed to further improve the robustness of QQ against noise and the reconstruction speed¹³.

The current QQ optimization assumes Gaussian noise for both QSM and mGRE magnitude, which may not be valid especially in low SNR regions, e.g., stroke lesions, due to QSM estimation by multi nonlinear steps from mGRE phase signal with non-Gaussian noise and Rician noise in mGRE magnitude. We formulated a novel QQ using the assumption of Gaussian noise in multi-echo complex mGRE data with a deep learning solver (mcQQ-NET) and compared it with the current deep learning based QQ (QQ-NET) in simulation and ischemic stroke patients.

### 2.2 THEORY AND METHODS

Multi-echo complex QQ (mcQQ). The mcQQ model combines the QSM-based and qBOLD based OEF mapping methods, according to known physical laws governing dephasing by dioxyheme iron in capillaries, venules and veins, using a nonlinear complex formulation to estimate OEF=1−Y/Yₐ with venous oxygenation (Y) and arterial oxygenation (Yₐ=0.98).

$${arg min}_{Y , v , {R}_{2} , {S}_{0} , {χ}_{n}} \text{} \{ {\sum }_{j} {‖ {S}_{j} - {F}_{qBOLD} ( {S}_{0} , Y , v , {R}_{2} , {χ}_{n} , {t}_{j} ) {e}^{i {ω}_{0} {t}_{j} d * {F}_{QSM} ( Y , v , {χ}_{n} )} ‖}_{2}^{2} + R \} , \qquad [ 3 ]$$

Here, Sⱼ is the complex signal at the j′th echo with removal of the initial phase and background phase, ω₀ Larmor frequency, d the dipole kernel, * the convolution operator. For robust parameter determination, we imposed total variation on Y(R). The QSM-based model separates voxel-wise susceptibility into the contribution of deoxy-hemoglobin in vasculature, venous blood, i.e., OEF effect, and non-blood neural tissue susceptibility

$$( {χ}_{n} ) \cdot {F}_{QSM} ( Y , v , \text{} {χ}_{n} ) = [ \frac{{χ}_{ba}}{α} + {ψ}_{Hb} \cdot {Δχ}_{Hb} \cdot ( - Y + \frac{1 - ( 1 - α ) \cdot {Y}_{a}}{α} ) ] \cdot v + ( 1 - \frac{v}{α} ) \cdot {χ}_{n}$$

where χ_{ba}=−108.3 ppb the fully oxygenated blood susceptibility assuming tissue hematocrit Hct=0.357, α=0.77 the ratio between the venous blood volume (ν) and total blood volume, ψ_{Hb}=0.0909 the hemoglobin volume fraction with Hct=0.357, Δχ_{Hb}=12522 ppb the susceptibility difference between deoxy- and oxyhemoglobin.

The vasculature is modeled as a collection of cylinders for computing deoxyheme iron caused spin dephasing, which forms the qBOLD model. Accordingly the OEF effect on the mGRE magnitude is described by F_{qBOLD}(S₀, R₂, Y, ν, χₙ, tⱼ)=S₀·e^{-R}^{₂}^{·t}^{ⱼ}·F_{BOLD}(Y, ν, χₙ, tⱼ)·G(tⱼ), where S₀ is signal intensity at t=0, R₂ is the transverse relaxation rate, F_{BOLD}(Y, ν, χₙ, t)=exp[−ν·fₛ(ωᵥ·t)] where fₛ is the signal decay by the presence of the blood vessel network and δω is the characteristic frequency due to the susceptibility difference between deoxygenated blood and the surrounding tissue:

$${ω}_{v} ( Y , {χ}_{n} ) = \frac{1}{3} \cdot γ \cdot {B}_{0} .$$

[ψ_{Hb}≠Δχ_{Hb}·(1−Y)+χ_{ba}−χₙ] with γ=267.51 rad·s⁻¹T^{31 1} the gyromagnetic ratio, and B₀ the main magnetic field strength. G(tⱼ) is the macroscopic field inhomogeneity contribution to mGRE signal decay.

Eq.3 is solved using a deep neural network, mcQQ-NET, consisting of 2 fully 4D (3D with multiple channels corresponding to the model parameters S₀, Y, ν, R₂, χₙ) convolutional subnetworks based on U-net, each for mGRE phase and magnitude input, respectively (FIG. 1). mcQQ-NET was trained with simulated data based on the QQ solution as ground truth after adding gaussian noise to the simulated complex mGRE. The loss function was weighted sum of 1) L1 difference between the truth and output of mcQQ, 2) model loss (Eq. 1), and 3) L1 difference of Y spatial gradient. mcQQ-NET was implemented using Pytorch 1.4.0 with performing optimization by ADAM, and training was stopped at 240 epochs as the validation loss became stable.

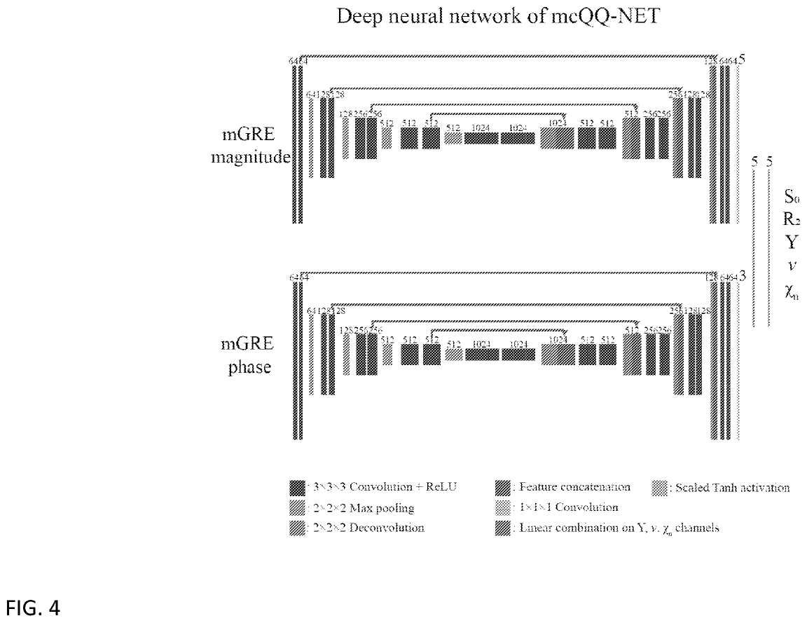

Validation. mcQQ was compared with the prior QQ solved using deep neural network¹³ in 1) simulated stroke brains (FIG. 2) and 2) 30 real ischemic stroke patients in which 3D mGRE (0.47×0.47×2.0 mm³ voxel size, TE₁/ΔTE/TE₈=4.5/5/39.5 ms, TR=42.8 ms) and DWI (0.94×0.94×3.2 mm³ voxel size, 0, 1000 s/mm² b-values) was acquired. The stroke patients were classified into 2 groups based on the time interval between stroke onset and MRI scan: acute (6-24 hours, N=5) and subacute (1-14 days, N=25) phase (FIGS. 3 and 4). Five-fold cross-validation was performed in all experiments to ensure no overlap between training and test data.

### 2.3 RESULTS

mcQQ showed more accurate OEF in simulation with a smaller mean error compared to QQ (FIG. 2) with better depicting low lesion OEF abnormalities (orange arrows). In the stroke patients, mcQQ showed improved spatial overlap between low OEF regions and DWI-defined lesions in the subacute phase (FIG. 3). In FIG. 4, mcQQ provided significantly lower OEF ratio between lesion and its contralateral normal tissue than QQ in the subacute phase, 0.71±0.15 vs. 0.61±0.18 (p<0.001, Wilcoxon signed rank test), but similar OEF ratio in the acute phase, 0.96+0.08 vs. 0.96+0.09 (p=1.000).

### 2.4 CONSIDERATIONS

This study demonstrated the feasibility of a multi-echo complex signal modeling approach for QQ (mcQQ). The improved physics model led to 1) improved OEF accuracy in simulation (FIG. 2), especially in the low OEF lesions, and 2) better depiction of OEF abnormalities in subacute stroke patients (FIGS. 3 and 4). The more realistic data noise consideration in mcQQ is particularly beneficial in low SNR regions. With improved sensitivity to OEF abnormality, mcQQ can be readily applied to investigate tissue variability in neurologic disorders including Alzheimer's disease and multiple sclerosis.

Overall methodology for this invention is based on the following consideration. Realistic or faithful modeling of MRI signal leads to accurate estimation of underlying biophysical parameters. We have demonstrated improvement of mcQQ with proper Gaussian noise model in multiecho complex data domain over the prior QQ with problematic Gaussian noise in magnitude and QSM. While model complexity may increase prediction or test error in classical optimization, model complexity faithful to underlying processes decreases test error in deep learning. Accurate signal simulations have become increasingly used in training deep neural network (DNN) to solve complex inverse problems, in a wide range of complex systems, including climate science, quantum chemistry, biological sciences, and biomedical imaging. Accordingly, we develop faithful mGRE signal model for extracting accurately OEF from the mGRE signal measurements.

Weakness in the prior research is that the closed-form signal model in Eq.3 omits the myelin component important for white matter tracts and is accurate only in limited situations. Our invention is to improve mGRE signal model through 1) a comprehensive closed form and then 2) a general digital twin. The comprehensive closed form provides a lower-hanging-fruit of improving Eq.3 with a myelin term and prepares us to develop the general digital twin that removes assumptions required by the closed form. We outline the corresponding mathematic details in the following sections.

Eq.3 can be extended according to known physical laws governing spin dephasing in magnetic fields to 3-compartment signal model for deoxyheme in vasculature, myelin and diffuse neural background that differentially affect mGRE signal. The above mGRE complex signal model in Eq.3 accounts for deoxyheme iron in capillaries and veins by modeling them as randomly-oriented paramagnetic cylinders, but ignores the diamagnetic myelin of lipid bilayers that are the major components of white matter. The dia- and para-magnetic sources in QSM can be separated solely based on mGRE data, as they contribute oppositely to susceptibility measured on QSM and additively to magnitude signal decay. Both diamagnetic myelin and paramagnetic vasculature are modeled in cylindrical shapes that affect mGRE signal very differentially from how diffuse background neural susceptibility affects mGRE signal. Specifically, Eq.3 can be extended to include myelin effects on mGRE signal, which can be modeled as parallel-oriented diamagnetic cylinders to estimate myelin quantity and orientation. For accurate OEF in white matter, the mGRE signal model should include effects of diamagnetic cylinders and sum over all components in a voxel, signal from water outside vasculature and myelin, signal from water within vasculature and signal from ware in myelin. Accordingly, we update Eq.3 with its terms as,

$${F}_{QSM} ( v , ℴ , {χ}_{n} , l , α ) = v ℴ \text{} {ψ}_{Hb} Δ {χ}_{Hb} + \frac{v}{a} {χ}_{ba} + ( 1 - \frac{v}{α} ) ( {lX}_{m} {sin}^{2} α + ( 1 - l ) {χ}_{n} ) \qquad [ 4 ]$$

$${Q}_{j} ( ℴ , v , {χ}_{n} , {m}_{0} , {ϕ}_{0} , {R}_{2} , l , α ) = {m}_{0} {e}^{- i {ϕ}_{0}} {e}^{- {R}_{2} {t}_{j}} ( \begin{matrix}{e}^{- i {ω}_{0} {t}_{j} d * {F}_{QSM} ( ℴ , v , {χ}_{n} , l , α )} ( 1 - v - l ) {e}^{- {vf}_{r} ( {ω}_{v} {t}_{j} )} {e}^{- {lf}_{‖} ( {ω}_{1} {t}_{j} )} + \\ {vS}_{s} ( {ω}_{v} {t}_{j} ) + {lS}_{h} ( {ω}_{1} {t}_{j} )\end{matrix} ) g ( {t}_{j} ) ,$$

$${arg min}_{ℴ , v , {χ}_{n} , {s}^{0} , {R}_{2} , l , α} {\sum }_{j = 1}^{N} {‖ {S}_{j} - {Q}_{j} ( ℴ , v , {χ}_{n} , {m}_{0} , {ϕ}_{0} , {R}_{2} , l , α ) ‖}_{2}^{2} + R .$$

Here l is the myelin lipid volume fraction, a is the angle of myelin orientation relative to the BO direction to account for myelin magnetic anisotropy with Xₘ=½(χₘ^{∥}−χₘ^{⊥})=−0.1115 ppm as half of the difference of the volume magnetic susceptibilities parallel and perpendicular to the lipid carbon chains in normal human white matter, ω_{|}=⅓ω₀(Xₘ sin² α−χₙ) myelin resonance frequency relative to diffuse background χₙ, Sₛ is the solid cylinder signal function, and Sn is the hollow cylinder signal function.

Eq.3 can be extended according to known physical laws governing spin dephasing in magnetic fields to 3-compartment signal model for paramagnetic deoxyheme iron in vasculature, diffuse paramagnetic iron, and diamagnetic that differentially affect mGRE signal. This is to incorporate Eq. 1 of positive and negative susceptibility sources into Eq.3. Then Eq.3 is updated to

$${F}_{QSM} ( v , ℴ , {χ}_{n} , {χ}_{n -} ) = v ℴ \text{} {ψ}_{Hb} Δ {χ}_{Hb} + \frac{v}{a} {χ}_{ba} + ( 1 - \frac{v}{α} ) ( {X}_{n +} + {χ}_{n -} ) \qquad [ 4 a ]$$

$${Q}_{j} ( ℴ , v , {χ}_{n +} , {m}_{0} , {ϕ}_{0} , {R}_{2} ) = {m}_{0} {e}^{- i {ϕ}_{0}} {e}^{- h ( {χ}_{n +} - {χ}_{n -} ) {t}_{j}} ( \begin{matrix}{e}^{- i {ω}_{0} {t}_{j} d * {F}_{QSM} ( ℴ , v , {χ}_{n +} , {χ}_{n -} )} ( 1 - v ) {e}^{- {vf}_{r} ( {ω}_{v} {t}_{j} )} + \\ {vS}_{s} ( {ω}_{v} {t}_{j} )\end{matrix} ) g ( {t}_{j} ) ,$$

$${arg min}_{ℴ , v , {χ}_{n} , {s}^{0} , {R}_{2} , l , α} {\sum }_{j = 1}^{N} {‖ {S}_{j} - {Q}_{j} ( ℴ , v , {χ}_{n} , {m}_{0} , {ϕ}_{0} , {R}_{2} , l , α ) ‖}_{2}^{2} + R .$$

In addition to determining This extension allows determination of diffuse paramagnetic iron, which is largely located in neurons and microglia in brain tissue and which is useful for characterizing neuroinflammation and neurodegeneration.

Eq.3 can be extended according to known physical laws governing spin dephasing in magnetic fields to 4-compartment signal model for paramagnetic deoxyheme iron in cylindrical vasculature, diffuse paramagnetic iron, diamagnetic myelin in cylindrical shapes, and diffuse diamagnetic source that differentially affect mGRE signal. This is to integrate Eqs. 4&4a together. Then Eq. 3 is updated to use the following

$$[ 4 b ]$$

$${Q}_{j} ( ℴ , v , {χ}_{n} , {m}_{0} , {ϕ}_{0} , {R}_{2} , l , α ) = {m}_{0} {e}^{- i {ϕ}_{0}} \text{} {e}^{- h ( {χ}_{n +} - {χ}_{n -} ) {t}_{j}} ( \begin{matrix}{e}^{- i {ω}_{0} {t}_{j} d * {F}_{QSM} ( ℴ , v , {χ}_{n} , l , α )} ( 1 - v - l ) \\ {e}^{- {vf}_{r} ( {ω}_{v} {t}_{j} )} {e}^{- {lf}_{‖} ( {ω}_{1} {t}_{j} )} + {vS}_{s} ( {ω}_{v} {t}_{j} ) + {lS}_{h} ( {ω}_{1} {t}_{j} )\end{matrix} ) g ( {t}_{j} ) ,$$

Other equation components are kept the same with χₙ=χₙ₊+χₙ₋.

Inversion with deep neural network (DNN). The inverse problem Eq.4 is solved using DNN (FIG. 4), which can provide fast reconstruction. A DNN can implicitly formulate denoising regularization through its large number of network weights and can solve nonconvex optimization problems with its stochastic gradient descent (SGD). DNN has been shown to be useful for multiparametric mapping of relaxation and diffusion tensor in MR fingerprinting and ASL perfusion. We have pioneered using known physics laws to reduce the generalization errors of a trained DNN as in our fidelity imposed network edit (FINE) and to solve a nonconvex problem without training. Accordingly, we investigate a novel deep neural network (DNN) formulation and solver of the nonconvex problem of Eq.4. Specifically, we investigate 1) no training DNN (NTD) using a many layer 3D U-net type network Ψ(S; θ)={σ, ∇, χₙ, m₀, ϕ₀, R₂, l, α} with a cost function E_{NTD}=Σⱼ∥S(tⱼ)−Qⱼ(Ψ(S;θ)∥₂², which is Eq.4 regularization represented by θ network weights; 2) accelerating NTD reconstruction while preserving accuracy by combining FINE and unsupervised training DNN (UTD) with a cost function E_{UTD}=Σᵢ₌₁^{M} Σⱼ∥Sᵢ(tⱼ)−Qⱼ(Ψ(Sᵢ; θ))∥₂².

Training data {Sᵢ} are simulated according to the forward problem signal equation (signal model in Eq. 4 with Gaussian noise added) under the full pathophysiological ranges of all parameters as demonstrated in our prior mGRE signal simulation work. Various echo times and resolutions are simulated as metadata to train DNN (U-net+weight predictor) for processing mGRE data acquired at a range of imaging parameters.

### 3. Brain Tissue Digital Twin

In this section, we present an invention, a brain tissue digital twin at micron resolution. The static dephasing model that leads to the closed form expression for the signal model in Eqs. 3 & 4 assumes no spin diffusion and simplifies calculation complexities, which may lead to errors in interpreting mGRE signals. These assumptions/simplifications can be avoided/reduced for more accurate expression of the mGRE signal model in the non-closed or digital form. In the modern computational method of fingerprinting or general deep learning, the high accuracy digital simulation of tissue MRI signal is very useful for developing algorithms to map tissue biophysics parameters. In the absence of ground truth for validating quantitative images of biophysical parameters, digital twin serves as an in silico validation framework. Accordingly, we have developed a brain tissue digital twin at the micron resolution with the following realistic features: 1) microscopic vasculature and myelin tracts in diffuse neural susceptibility source, 2) water spin diffusion via Monte Carlo simulation, 3) oxygen extraction at capillaries, and 4) precision drug delivery.

### 3.1 VASCULATURE

We have generated vascular digital twin at the micron resolution for the kidney, the liver and the brain tumor. The constrained constructive optimization (CCO) is used to generate realistic 3D vessel trees with bifurcations according to mass conservation and Murphy's power law by iteratively connecting new terminal nodes. The CCO is an optimization framework that balances the root arterial flow with the terminal branch outlets and allows vascular growth adaptive for incorporating patient-specific anatomy such as tumor vascular geometry and density. We have refined CCO features according to patient specific imaging information, including optimization cost as the total vascular space at each iteration, new terminal location seeding in each iteration, and the exponent in Murray's power law. The realistic capillary distribution in human brain (peak at 8 mm diameter and approximately Gaussian) is input into CCO for generating terminal capillaries. The small arteries and veins are constrained to be perpendicular to the cortical surface. The blood flow in the microvasculature is generated according to Poiseuille flow.

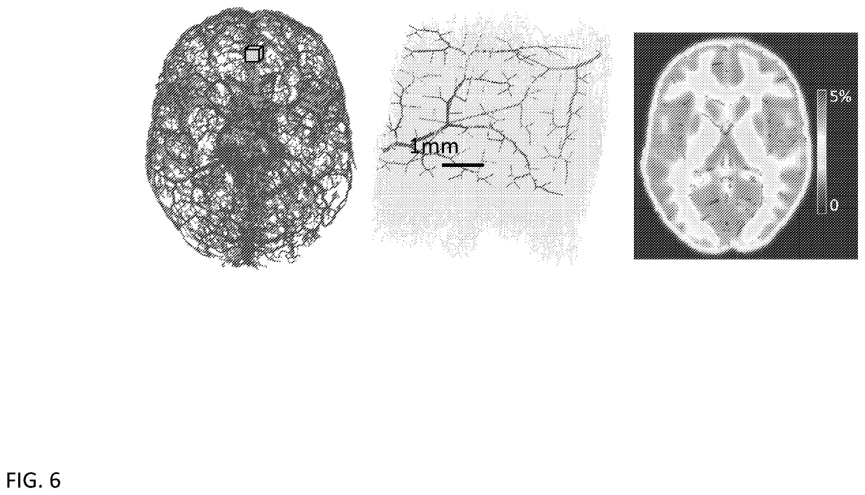

FIG. 6 shows a specific brain vascular digital twin that we have generated using the CCO method. We start from arteries extracted from time of flight (TOF) image and veins extracted from susceptibility weighted imaging (SWI) images. We divide the brain volume into 10 μm grid, set the perfusion for gray matter to 60 mL/100 g/min and the perfusion for white matter to 40 mL/100 g/min, and use the minimal total vascular volume and cubic vessel diameter flow constraints in the CCO method. We use the same CCO method to generate separately the arterial vasculature starting from TOF and the venous vasculature starting from SWI. The construction pipeline based on CCO method is as follows. First, we define the following constants: Terminal points—Outlet of the vascular tree, and will be connected to capillaries in the next step; d₁=5m—The maximum length of vessel segment; Nₐ=3, —Number of candidate segments that a terminal point will be connected to; ∈(P), —Grid that contains vessel point P; F(∈)—Perfusion of grid ∈; and we define the following three criteria: C₁(P)—No other terminal exists inside the same grid P belongs to; C₂(S, P)—The distance between vessel segment S and P is smaller than d₁; C₃(P₁, P₂)—The distance between P₁ and P₂ is smaller than d₁, and P₁ and P₂ are not in the same grid. Then the algorithm for the arterial tree can be written as
Do
   Randomly draw a candidate terminal point P₁ inside the volume such that C₁(P₁) is true.
      Find Nₐ vessel segments Sᵢ closest to P₁ such that C₂(Sᵢ, P₁) is true.
      For each vessel segment Sᵢ found in line 4
         Set flow of terminal point F₁ as F(∈(P₁)) and connect to Sᵢ.
         Update the flow of vascular tree.
         Determine the optimal connecting point P₂ from P₁ to Sᵢ by minimizing the total vessel tree volume.
      Traverse possible vessel segment Sᵢ and connect P₁ to the vessel segment that gives the minimal total vessel tree volume.
      Update the flow of vessel tree.
Until there is no more terminal point P such that C₁(P) is true.

While minimizing the total vasculature volume, we assumed the cubic of vessel diameter is proportional to flow, with the proportionality factor set to one, since we are interested in the relative vessel volume only. The venous vasculature was constructed separately using the same method, and representative generated arterial and venous vasculatures are shown in FIG. 6.

### 3.2 MYELIN TRACTS AND NONUNIFORM DIFFUSE NEURAL SUSCEPTIBILITY SOURCE

We introduce the white matter (WM) myelin compartment into the digital twin. The WM myelin density in the digital twin can be defined according to myelin water fraction, and the WM myelin fiber cylinder orientation can be defined according to diffusion tensor imaging. WM myelin cylinders are treated as hollow cylinders (1 μm myelin diameter, 0.8 g-ratio): The hollow cylinder wall is composed of lipid bilayers that have an anisotropic magnetic susceptibility described by a tensor with a radially oriented principal axis; The myelin water consists of axonal water in the inner cylinder hole and the water in the myelin sheath wall with a reduced T2 relaxation time and spin density and undergoes chemical shift/exchange. The myelin lipid volume fraction l estimated in the above Eq.4, which determines the myelin water fraction (MWF) and can be determined from MWF, is used to assign myelin cylinder density and avoid microvascular structures in space.

Similarly, the diffuse neural susceptibility source χₙ estimated in the above (likely dominated by iron stored in ferritin in neural cells) is imposed in the extravascular extramyelin space for background.

### 3.3 OXYGEN EXTRACTION FRACTION AND MRI SIGNAL WITH DIFFUSION EFFECTS

We introduce oxygen extraction simulation into the vascular digital twin in order to simulate oxygen extraction in healthy functions and in various diseases. Oxygen extraction occurs during the transits of red blood cells through capillaries according to the Bohr-Kety-Crone-Renkin flow diffusion equation. The OEF over a single capillary σ_{c} is the difference in blood oxygen concentrations at entrance C(0) and exit C(1) scaled by C(0) with blood oxygen loss along the capillary proportional to intracapillary oxygen partial pressure as determined by the Hill equation

$${ℴ}_{c} = 1 - \frac{C ( 1 )}{C ( 0 )} , \frac{dC ( x )}{dx} = - \frac{D \cdot {P}_{50}}{f} {( \frac{C ( x )}{B - C ( x )} )}^{1 / h} , \qquad ( 1 )$$

where x is position along the capillary scaled to the capillary length, f blood flow, D=0.0048 mL/mmHg/mL/min effective oxygen diffusivity, P₅₀=26 mmHg oxygen pressure for half saturation and B=0.1943 mL/mL maximum oxygen bound to hemoglobin. We can adjust blood flow to obtain various OEF in the brain digital twin. We can change effective oxygen diffusivity, oxygen bound to hemoglobin and blood flow to obtain pathological OEF values in localized lesions in the brain digital twin.

The existing analytical models of diffusion effects on MRI signal include ignoring diffusion (static dephasing regime), linear local field approximation, Gaussian phase approximation, and strong collision approximation. However, these models are only accurate in the limits of diffusion under which they were derived, and none of them is accurate for the whole physiological range of blood vessels radii and blood oxygenation levels, and all of them have systematic qBOLD errors caused by the approximations of these models. The Monte Carlo methods can be used to simulate the qBOLD effects in mGRE signal by diffusing water molecules in the presence of long, cylindrical blood vessels and myelin, and can significantly improve the qBOLD accuracy over a range of blood oxygenation levels and blood vessel radii than existing analytical models for extracting physiological parameters of the blood vessel network from experimental data in BOLD-based experiments. Accordingly, we use Monte Carlo simulation to generate digital form of qBOLD effects in mGRE complex signal.

Water protons in this brain digital twin consist of 4 compartments: vascular compartment, myelin axon hole compartment, myelin lipid wall compartment, and the main extravascular extramyelin (EVEM) compartment. The R₂ value differ among these compartments. At each time-point, the protons move in each dimension by a step that is randomly sampled from the distribution N(0, 2Dδt) where D is the diffusion coefficient (˜2 μm²/ms except 0 in the myelin lipid wall) and δt is the time step (˜25 μs or sufficiently small). If the step brought a proton into contact with a vessel it was mirror-reflected from the surface, since the vessels are approximately impermeable for MRI signal formation. mGRE signal is calculated according to spin dephasing effects and spin diffuse effects averaging over all protons in all compartments,

$${Q}_{j} = \frac{1}{N} {\sum }_{p = 1}^{N} {e}^{- i {ϕ}_{p}} {e}^{- {R}_{2} \cdot {t}_{j}} + ε , {ϕ}_{p} = {\sum }_{m = 1}^{j} {\sum }_{n = 1}^{{N}_{s}} {ω}_{mpn} δ t , \qquad [ 5 ]$$

where ωₘₚₙ is the proton precession frequency contribution from n susceptibility source (vascular cylinders and myelin cylinders, a total Nₛ) on proton p (of a total N) at time-point m (of a total tⱼ/δt at time tⱼ) according to dipole fields expressed in off-resonance,

$$δω = δχ \frac{{ω}_{o}}{2} {( \frac{a}{ρ} )}^{2} cos 2 φ {sin}^{2} θ$$

caused by a long solid cylinder of radius a and δχ susceptibility relative to background at a distance ρ with azimuthal/polar angle φ/θ, and ε is Gaussian noise. The simulation can be sped up by focusing on susceptibility sources within 3 mm of a proton and by skipping other steps of little changes.

### 3.4 INVERSION WITH A DEEP NEURAL NETWORK (DNN)

Digital twin data may be summarized by an approximate phenomenological model. A better option of no approximation is to store the digital twin data information in a deep neural network (DNN) or to train a DNN. Specifically, the DNN in the above Section 2 are trained using digital twin data. The vascular and myelin structures in the whole brain volume (˜1000 cm³) are defined by a set of cylinders, which can be stored in hard disk. These vascular and myelin structures in an image voxel are retrieved for simulation at 1 μm resolution in the forward problem of computing field and block-accessed recursively. Many brain digital twins are generated from different subjects' MRI data. For a brain digital twin noted by index i, mGRE signal and biophysics parameters in Eq.3a at the micron resolution are down-sampled to imaging resolution as Sᵢ and Ψ_{ref}(Sᵢ)={σ, ν, χₙ, m₀, ϕ₀, R₂, l, α} for supervised training of the DNN (STD) with a cost function E_{STD}=Σᵢ₌₁^{M}∥Ψ_{ref}(Sᵢ)−Ψ(Sᵢ; θ)∥₁ for a training set of M brain digital twins. E_{STD} may include other regularizations such as tissue structure preservation with image gradient operations as we have demonstrated in QQ-NET. As in the above Section 2, various echo times and resolutions are simulated as metadata to train DNN (U-net+weight predictor) for processing mGRE data acquired at a range of imaging parameters. This digital twin (DT) trained DNN are used to extract from mGRE data OEF maps, which are denoted as OEF_{DT}.

### 3.5 QUANTITATIVE SUSCEPTIBILITY MAPPING, TRANSPORT COMPUTATION AND PRECISION DRUG DELIVERY

The scientific premise is that the transport of contrast agents mapped in contrast enhanced MRI can be translated into predicting distribution of transarterial drug delivery in the same vascular system according to the underlying transport physics.

For precision medicine with targeted drug delivery approach, improving match between drug and disease distributions has been identified as a major unmet need. Intravascular targeted delivery is commonly practiced in interventional angiographic suites, including TARE that delivers radiotherapy microspheres through feeding hepatic arteries into liver tumors. The strength of the prior research is that computational fluid dynamic (CFD) models for hepatic artery tree and particle transport have recently been developed for precision treatment planning to improve clinical outcomes. The weakness of the prior research is that there is only patient's macrovascular anatomy input into these CFD models, and no input of patient's microvascular information causes use of many parameters 12 not individualized for patients.

Specifically, pretreatment imaging C-Arm cone-beam CT with high resolution mapping of hepatic arteries can be input into CFD modeling for estimating microsphere distribution and is used to guide TARE practice. However, CT voxels are still macroscopic, and the smallest vessel diameter segmentable on CT is about 500 μm that is about 20 times the diameter of Y90 microspheres (diameter about 25 μm). Consequently, the distribution of small vessels and capillaries is modeled according to lumped elements or other assumptions, and also regional blood flow distribution is also generated from general assumptions, both without input of patient-specific distributional information. Because diseases invariably cause inhomogeneous perturbation from general model assumptions, patient-specific information about small vessels and capillaries and regional blood flow would improve CFD fidelity with the underlying pathophysiology in a patient.

Patient-specific microvasculature information can be obtained from pretreatment imaging and be applied to personalized precision computation modeling of microsphere delivery. MRI, in addition to CT, from pretreatment imaging can be used to acquire quantitative distributional information about small vessels and capillaries and about regional blood flow. Radii of small vessels and capillaries in tissue including tumors can be mapped from vessel size imaging (VSI), which is based on changes in R2 and R2* caused by paramagnetic contrast agent in vessels maps. Regional microvascular blood volume and blood flow can be mapped from quantitative perfusion imaging (QPI) using dynamic and equilibrium imaging of paramagnetic contrast agents. An intravascular iron oxide nanoparticle agent (ferumoxytol) can be used for both VSI and QPI: spacetime resolved 4D imaging during the dynamic phase of initial injection provides data for QPI. Combined spin echo and gradient echo imaging during the equilibrium state provides data for VSI.

We have also pioneered contrast enhanced MR angiography, quantitative susceptibility mapping (QSM) that is well suited for quantifying paramagnetic contrast agents, and ferumoxytol (Fe) enhanced vascular MRI. To overcome the problem of using a global arterial input function to all voxels in current QPI, we have recently developed a novel approach of including the mass flux gradient in the QPI formulation to rigorously follow the law of mass transport physics, which we term as quantitative transport mapping (QTM). Accordingly, we apply these experiences and insights of imaging contrast agent transport in MRI to achieving microvascular information for addressing the weakness of the prior research. We develop for TARE, 1) VSI, 2) hepatic blood volume (HBV) mapping using QSM processing of VSI gradient echo phase data, 3) hepatic blood flow (HBF) mapping using QTM, 4) integrating VSI, HBV, and HBF into the CFD model with vascular tree branching to 25 μm level to predict the delivered microsphere distribution in TARE. Accordingly, this invention includes 1) methods to map hepatic vasculature and flow at macro- and micro-levels using vascularity-sensitive MRI acquisitions, an intravascular paramagnetic contrast agent and data modeling according to magnetism and transport physics. We acquire MRI data during the dynamic circulation following Fe injection and during Fe equilibrium circulation, obtain Fe concentration ([Fe]) using QSM, obtain HBV from equilibrium [Fe], HBF using QTM from dynamic [Fe], microvascular size using VSI. 2) Develop CFD model with microvascular tree branching using intravascular paramagnetic contrast agent transport imaging to predict quantitative embolization delivery. We develop hepatic CFD model with microvasculature from VSI, HBV and HBF, and microvasculature from 4D flow and CTA. 3) Validation of hepatic CFD model for microsphere delivery distribution in TARE using posttreatment Y90 PET.

Quantitative susceptibility mapping for estimating tissue regional blood volume from susceptibility affected imaging according to magnetism physics. Reginal blood volume V(r) consists of macrovascular volume V_{M}(r) and microvascular volume V_{μ}(r). Current estimation based on modeling the change of gradient echo signal decay rate ΔR₂*(r) caused by an intravascular paramagnetic agent during its equilibrium circulation, which causes a vascular susceptibility change Δχᵥ, only allows sensitizing V_{μ}(r). The static dephasing calculation at a vascular approximation of randomly-oriented dilute small cylinders leads to: ΔR₂*(r)=4π/3γΔχ_{ν}B₀V_{μ}(r). Consequently, there are two difficulties with this current approach. 1) The calculation based on effects of small vessels and capillaries does not account for effects from large vessels. 2) The vascular susceptibility change Δχ_{ν} must be estimated from additional measurements of longitudinal relaxation rate change ΔR₁ or from empirical estimation of total blood volume. Both these concerns can be readily addressed using QSM: Δχ_{ν} can be directly measured at voxels of full vascular space, such as a region of aorta. The regional blood volume as a fraction of voxel volume is the simply a scaling factor for measured susceptibility change by the intravascular contrast agent: Δχ=Δχ_{ν}V(r), or V(r)=Δχ/Δχ_{ν}.

Quantitative determination of microsphere delivery distribution at microvasculature level according to MRI estimated microvascular geometry and flow. The major goal of TARE treatment of liver tumor is to deliver adequately high concentration dose in the tumor, while sparing the surrounding parenchyma. Computational fluid dynamics (CFD) model for patient-specific simulation of entire hepatic arterial tree, based on liver, tumors, and arteries segmentation on patient's CT of hepatic arteries is limited to arteries down to a diameter of 500 μm. Microvascular network information of the liver with tumor is needed to extend the CFD dose model to 25 μm. The microvascular size and flow mapping from MRI as described in this project can enable patient-specific CFD dose model at 25 μm. The treating physician can use this patient-specific CFD modeling of microspheres distribution to identify the optimal catheter placing in treatment planning.
1) MRI data acquisition for vascular geometry and flow characterization. For tissue regional hepatic blood flow mapping, we perform dynamic susceptibility contrast MRI during the dynamic phase immediately following Fe injection. For regional hepatic blood volume mapping, we perform multiecho gradient echo imaging before and substantially after Fe injection with phase saved for QSM processing. For microvascular size index mapping, we perform diffusion weighted imaging (DWI) before Fe injection, multiecho spin echo imaging before and substantially after Fe injection, in addition to gradient echo imaging. For macrovascular geometry and flow, we perform 4D flow imaging in MRI with the benefit of contrast enhancement and utilize available 3D CT angiogram.

Preinjection imaging: 1) Diffusion-weighted imaging: DWI with b=0, 200, 800 s/mm2 for each of the 3 principal magnet axes, TR ˜3000 ms, TE ˜95 ms, eight averages, free-breathing) were also acquired, and used to calculate apparent diffusion coefficient (ADC) maps using monoexponential curve fitting. 2) R2 spin echo imaging. A navigator gated dual-echo 3D fast spin-echo (FSE) T2-weighted sequence (pulse repetition time (TR) 2600 ms, echo times (TE) 6 and 96 ms, echo train length 15) was used to derive maps of the estimated transverse relaxation rate, R2. 3) R2*+QSM gradient echo imaging. A navigator gated multiecho gradient-echo 3D sequence (TR 170 ms, TE 3.4-39 ms). In phase echoes for robust fat-water separation and QSM. Additional imaging included anatomical scans (fast imaging with steady-state precession, FISP/FIESTA), T1-weighted and multi-flip angle T1-mapping scans. The total scan time was approximately 30 min.

Dynamic Fe gradient echo imaging. A 3 mg Fe/kg dose of ferumoxytol diluted 1:1 with saline is injected at 2 mL/s using a power injector, followed by a saline flush. This dose, less than half of the full therapeutic dose, is chosen to be within the lower range of literature reported Fe doses 33, 34 to facilitate repeat studies. Data is acquired using a multiecho 3D spoiled gradient echo golden angle interleaved spiral sequence during Fe dynamic phase to form 4D space-time resolved magnetic field reconstruction using the temporal resolution acceleration with constrained evolution reconstruction (TRACER): stack-of-spirals with variable density 4 echoes, self-gating based on z-profile, TR/TE_{first}/TEₗₐₛₜ=34.3/0.7/25.3 ms, echo spacing=8.2 ms, bandwidth=±125 kHz, flip angle=15°, FOV=40 cm, matrix size=256×180×40, slice thickness=3 mm, reconstruction voxel size=0.78×0.78×1.5 mm³, temporal frame very 0.5 s.

Postinjection imaging. 1) 4D flow imaging. Phase contrast MRA at proper hepatic artery, left hepatic artery and right hepatic artery. 4D flow MRI is performed at an axial volume covering the liver. Data are acquired with prospective respiratory navigator gating. Pulse sequence parameters were as follows: spatial resolution, 2.×2×3 mm; temporal resolution, 40 msec; field of view, 340×238×108 mm; TE/TR=2.6/5.0 msec; flip angle=15°; total scan time=12 minutes; and velocity sensitivity, 80 cm/sec in all three velocity-encoding directions. 2) R2 spin echo imaging and 3) R2*+QSM gradient echo imaging as in preinjection imaging are repeated.

Experiment—Patient Recruitment. Patients clinically indicated for 90Y TARE therapy are recruited for this study, including those being diagnosed with hepatocellular carcinoma, primary colorectal cancer and liver metastases from primary breast, thyroid, neoendocrine tumor, and pancreatic cancer. Contraindications consist of 1) TARE contraindications—patients with no-less-than 5% lung shunting or extrahepatic leakage and 2) MRI contraindications. Two weeks prior to Y90 TARE therapy, all patients receive 185 MBq Tc99m macroaggregated albumin (MAA) SPECT/CT (Infinia, GE Medical Systems, Milwaukee, WI). Patients are treated with either TheraSphere® (glass microspheres; BTG, London, UK; diameter range 20-30 μm; activity per sphere 150-2200 Bq) or SIR-Sphere® (resin microspheres; Sirtex Medical, Sydney, Australia; diameter range 20-60 μm with a median 32.5 μm; activity per sphere 65-140 Bq). The number of spheres per treatment is 1-2 million (less embolic effect) for TheraSpheres, and 40-60 millions (higher embolic load) for Sir-Spheres.
2) Hepatic 4D flow U(t,ξ), QSM hepatic blood volume ν(ξ), VSI microvasculature map R(ξ), quantitative transport mapping (QTM) to map regional hepatic blood flow u(ξ).

4D flow analysis. 4D flow MRI preprocessing includes corrections for noise, eddy currents, Maxwell terms, and velocity aliasing. 3D blood flow imaging of average velocity Ū(ξ)=<U(t,ξ)>ₜ and regional flow quantification are performed by using EnSight (CEI, Apex, NC). Time-resolved path lines are generated by using emitter planes placed in the common hepatic artery (HA). The resulting traces are color coded according to blood flow velocity. Net flow and peak velocity are calculated by using 8 analysis planes, which are manually positioned on anatomic landmarks: common HA, proper HA, left HA, cystic A, middle HA, right HA, anterior branch and posterior branch. These landmarks are registered with high resolution CT angiogram for upsampling flow data onto artery tree generation.

QSM regional hepatic blood volume ν(ξ). On the acquired equilibrium state gradient echo data, fat-water separation is performed according to the robust stochastic gradient descent in deep learning to determine water portion W, fat portion F, R₂* and field b. Then QSM with zero reference and shadow-resistance against motion, flow, noise and other errors is formulated as:

$${χ}^{*} = arg {min}_{χ} \frac{1}{2} {‖ {\sum }_{t} {e}^{- {iR}_{2}^{*} t} ( W + {Fe}^{- i {ω}_{F} t} ) ( {e}^{- i {ω}_{0} bt} - {e}^{- i {ω}_{0} ( d * χ ) t} ) ‖}_{2}^{2} + {λ}_{1} {‖ {M}_{e} ∇ χ ‖}_{1} + {λ}_{2} {‖ {M}_{u} ( χ - {χ}_{u} ) ‖}_{2}^{2}$$

Mₑ the binary edge mask derived from the magnitude image. For fat as uniform regions, χᵤ=Xₒₐ·ρ_{f}/ρₒₐ with ρ_{f} as the fat image and ρₒₐ the mean ρ_{f} value in the subcutaneous fat regions of assumed pure oleic acid. Human adipose tissue is composed largely of oleic acid with a volume susceptibility Xₒₐ=0.75 ppm. The pure fat may automatically be identified from maximal signal regions on the fat image. The L2 biological constraints in Eq. 8 may greatly improve QSM reconstruction SNR, as we have found in the piece-wise constant model. QSM is performed on data acquired before and after Fe injection. The total blood volume is determined with scaling factor Δχ_{ν} measured on a pure vascular region such as the aorta.

Vessel size imaging (VSI) analysis. Maps of D, R₂* and R₂ are generated from DWI, gradient echo and spin echo imaging data. Because the gradient echo decay rate R₂* is determined by field while spin echo decay rate R₂ by diffusion in field gradient, and the field outside a small cylindrical vessel filled with an intravascular paramagnetic agent Fe falls off with inverse radius squared, ΔR₂ and ΔR₂* induced by Fe provides differential sensitization of the small vessel radius. Detailed analysis of spin echo and gradient echo signal models lead to the following formulation of an average small vessel size index in μm:

$$ℜ = 0.867 {( {V}_{μ} D )}^{\frac{1}{2}} ( Δ {R}_{2}^{*} / Δ {R}_{2}^{3 / 2} ) = 0.867 {( \frac{3 D}{4 π γ Δ {χ}_{v} {B}_{0}} )}^{0.5} {( \frac{Δ {R}_{2}^{*}}{Δ {R}_{2}} )}^{1.5}$$

Quantitative transport mapping (QTM) of regional hepatic blood flow u(ξ). Dynamic Fe imaging is processed with QSM to determine spacetime resolved 4D [Fe] concentration c(r, t), as phase based QSM provides the benefit of overcoming difficulties in traditional estimation of contrast agent concentration from magnitude images. Then the 4D c(r, t) is processed using QTM to determine blood flow velocity according to the inverse problem of mass transport:

$$u = {min}_{u} {\sum }_{r = 1}^{N} [ {‖ {\partial }_{r} c + ∇ \cdot ( cu ) ‖}^{2} ] + λ ( {‖ ∇ u ‖}_{1} ) .$$

Here u(r)=[u^{x}(r), u^{y}(r), u^{z}(r)] the velocity vector of voxel r. The discrete temporal and spatial derivatives are difference operations, reducing the transport equation to linear algebraic form. An L1 regularization is added for denoising. Regularization parameter λ is chosen based on L-curve study.
3) Develop CFD model with microvascular tree branching using intravascular paramagnetic contrast agent transport imaging to predict quantitative embolization delivery. The tissue microvascular size index from VSI and flow velocity from QTM, in addition macrovascular geometry from CTA and velocity from 4D flow, can be incorporated into the CFD model for predicting drug delivery at tissue level.

Vascular tree generation according to imaging-estimated hepatic vasculature and flow information at macro- and micro-levels. A hepatic arterial tree, particularly detailed to 25 μm level in the liver segments containing tumors, is constructed for each patient. The 3D shape of the liver and the blood flows of the proper hepatic artery and major branches from MRI and CT data will be used as the starting place to grow the arterial network. There are several similarly iterative algorithms for generating realistic 3D vessel trees with bifurcations according to mass conservation and Murphy's power law by iteratively connecting new terminal nodes chosen from some volume. We follow the widely used constrained constructive optimization (CCO), which was used in our preliminary study. The CCO optimization framework offers the desired properties that the root arterial flow is balanced with the terminal branch outlets and that the vascular growth is adaptive for incorporating patient-specific anatomy such as tumor vascular geometry and density. We incorporate patient-specific macro- and micro-vasculature and flow information from MRI and CT (high resolution angiogram) into the CCO procedure in the following manner for generating the hepatic arterial tree (FIG. 18).

We have identified 3 features in the CCO iterative procedure that are generic and can be adjusted to patient specific information: 1) cost function for optimization is the total vascular space at each iteration; 2) new terminal location is randomly or pseudo randomly seeded in each iteration; 3) the exponent in Murray's power law is empirically selected between 2-3 without patient tissue specific input. We have addressed these issues as follows.
i) Adjust the cost function for optimization with a term for matching the average voxel velocity to QTM estimated velocity from MRI. The cost function at each branching iteration is the total blood volume the temporary tree at that iteration in the original CCO. This cost function can be adapted to oxygen demand and other purposes. We add a cost of matching the average velocity of the temporary tree branches at the surface of a voxel <u>ᵣ to the QTM velocity u(r) estimated from MRI according to least squares ∥<u>−u∥₂². The surface velocity average definition for QTM velocity is the vector flow into a voxel and naturally preserves flow balance in the CCO algorithm. We will finetune the cost function using a simulation approach where the ground truth is known.
ii) Modify new terminal location seeding probability according to regional blood volume estimated by QSM. This is straightforward in CCO algorithm and has been frequently practiced.
iii) Optimize Murray's exponent according to the estimated microvasculature size index (MSI) map from VSI. The terminal vessel radius at a high bifurcation level increases rapidly with the exponent with a very sensitive power law. An exponent varying with bifurcation level has recently been suggested for arterial network. We adapt the exponent to the MSI map from VSI by minimizing a cost ∥<R>−R∥₂², where <R>ᵣ is the microvascular size index computed from a temporary tree and R(ξ) MSI map from MRI

Quantitative microsphere delivery according to the generated vascular tree and computational fluid dynamics

Given the microvascular model, the intravascular blood velocity is governed by the Navier-Stokes equation and the continuity equation. The 3D microvascular network flow is simplified as a 1D non-linear network of cylindrical model. The flow rate Qᵢⱼ through each vessel segment (i, j) is related to the pressure drop (Pᵢ−Pⱼ) by Poiseuille's law,

$${Q}_{ij} = {G}_{ij} ( {P}_{i} - {P}_{j} )$$

$${G}_{ij} = \frac{π {R}_{ij}^{4}}{8 {μ}_{ij} {l}_{ij}}$$

where Pᵢ denotes the pressure at node i and Gᵢⱼ is the hydrodynamic conductance of segment (i,j), μᵢⱼ is the apparent viscosity of the blood, Rᵢⱼ and lᵢⱼ are the segment radius and length, respectively. Note that μᵢⱼ depends on the vessel diameter and hematocrit. The average velocity in a vessel segment is

$${\overline{v}}_{ij} = \frac{{Q}_{ij}}{π {R}_{ij}^{2}}$$

The parabolic velocity profile of the segment (i, j) is therefore expressed as

$${v}_{ij} ( r ) = 2 {\overline{v}}_{ij} ( 1 - {( \frac{r}{{R}_{ij}} )}^{2} )$$

where r is the radial distance from the center of the cylindrical vessel.

When microspheres are infused into the hepatic artery, they make their way through the arterial blood vessels to the tumor periphery, where they lodge in the capillaries that are narrower than their diameter (32 μm). Experiments show that microspheres aggregate in clusters (Campbell et al 2000, Pillai et al 1991). We predict the distribution of microspheres in the tumor according to the probability that a sphere reaches a particular branch. Since spheres are introduced at the initial segment of the vasculature tree the probability is 1 there. At each branching of the tree, the probability is divided between the daughters proportionately to the cross-sectional area of each daughter segment. At terminating branches the probability is that a sphere will lodge in that branch, so that value is placed at the end of that branch. The probabilities of terminating branches from all trees are divided by the number of trees and accumulated in voxels to produce a three-dimensional field of the microsphere probability distribution. The expected number of microspheres in a voxel is the probability of that voxel multiplied by the total number of microspheres that lodge in the tumor territory. Microsphere delivery according to CFD streamlines.

### 4. Device System Implementation

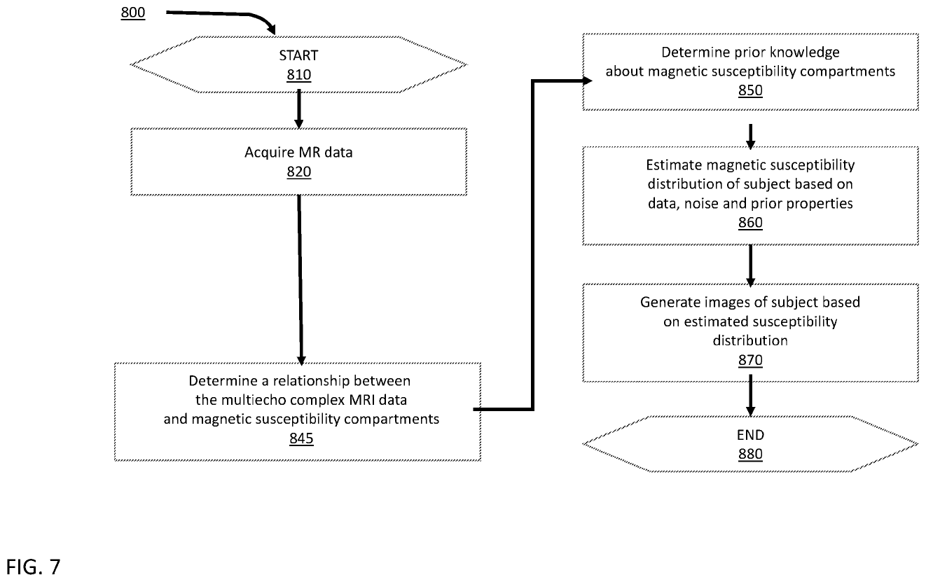

In some cases, one or more of the above quantitative susceptibility mapping techniques can be implemented using the process 800 shown in FIG. 7. As an example, the process 800 can be used to map the tissue magnetic susceptibility of a subject, such as a patient (or portion of a patient) or an experimental sample (e.g., an imaging phantom, a sample of material or tissue, and so forth).

In some implementations, the process 800 can be used to transform MRI signal data corresponding to a subject into susceptibility-based images that quantitatively depict the structure and/or composition of the subject. As an example, in some cases, the process 800 can be used to obtain MRI data corresponding a subject, and process this MR data to generate a quantitative susceptibility map of the subject. Using this quantitative susceptibility map, one or more susceptibility-based images of the subject can be generated and displayed to a user. The user can then use these images for diagnostic or experimental purposes, for example to investigate the structure and/or composition and/or function of the subject, and/or to diagnose various conditions or diseases based, and/or to treat various conditions or diseases based, at least in part, on the images. As the process 800 can result in susceptibility-based images having higher quality and/or accuracy compared to other susceptibility mapping techniques, implementations of the process 800 can be used to improve a user's understanding of a subject's structure and/or composition and/or function, and can be used to improve the accuracy of any resulting medical diagnoses or experimental analyses.

The process 800 begins by acquiring magnetic resonance (MR) data corresponding to a subject (step 810). In some cases, the MR data can correspond to a patient (e.g., the entirety of a patient or a portion of a patient, such as a particular portion to the patient's body). In some cases, the MR data can correspond to one or more samples (e.g., an imaging phantom, a sample of one or more materials, a sample of one or more types of tissue, and/or one or more other objects).

In some implementations, the MR data can be acquired using an MRI scanner using one or more suitable pulse sequences. As an example, in some cases, MR data can be acquired using a gradient echo sequence that acquires MR data at a single echo time or at multiple echo times (e.g., two, three, four, five, and so forth). Various scan parameters can be used. As another example, MR data can be obtained using a 3D multiecho spoiled gradient echo sequence on a 3T scanner using a pulse sequence that samples at different echo times (TE) (e.g., 4 TEs, such as 3.8, 4.3, 6.3 and 16.3 ms) in an interleaved manner, and with following imaging parameters: repetition time (TR)=22 ms; voxel size=0.5×0.5×0.5 mm³, matrix size=256×64×64; bandwidth (BW)=±31.25 kHz; flip angle=15°. As another example, MR data can be obtained using a 3D multi-gradient echo sequence on a 3T scanner using imaging parameters TE/ΔTE/#TE/TR/FA/BW/FOV/matrix size=5 ms/5 ms/8/50 ms/20°/±62.50 kHz/21.8×24×12.8 cm³/232×256×64. Although an example sequences and example parameters are described above, these are merely illustrative. In practice, other sequences and parameters can be used, depending on various factors (e.g., the size of region to be examined, a known or assumed range of susceptibility values of the subject, scan time considerations, device limitations, and so forth).

After acquiring MR data corresponding to a subject, the process 800 continues by determining a magnetic field based on the MR data (step 820). As noted above, in some cases, the MRI signal phase is affected by the magnetic field corresponding to the magnetic susceptibility distribution of the subject and the chemical shift of tissue components.

In some implementations as illustrated in FIG. 7, the magnetic field can be determined from the complex MR data by fitting the detected signal as a sum over tissue spectral components, where each component signal characterized by an exponent with a negative real part representing signal decay and an imaginary part representing phase dependent on the magnetic field and the chemical shift. This fitting for such a complex signal model can be performed on iteratively using numerical optimization. A robust initialization for numerical optimization may be estimated using deep neural network. After determining the magnetic field based on the MR data, the process 800 continues by determining a relationship between the magnetic field and magnetic susceptibility. As described above, in some implementations, the relationship between the magnetic field and magnetic susceptibility can be expressed as a relationship between the magnetic field distribution in space to the magnetic susceptibility at a given location. In some implementations as illustrated in FIG. 7, the magnetic field estimation step is bypassed, and the estimation of magnetic susceptibility map is performed directly on the multiecho complex data by forming a relationship between the multiecho complex data and the magnetic susceptibility compartments (step 845).

Then process 800 continues by determining prior knowledge about tissue magnetic susceptibility distribution (step 850). One estimation of tissue susceptibility distribution is to use R2* values derived from the magnitude signal. High R2* values can be used to identify high susceptibility regions such as hemorrhages for preconditioning to accelerate the convergence of numerical optimization. An example of preconditioning is to separate the whole image volume into region with high susceptibility (including hemorrhages, air regions and background) and region with normal tissue susceptibility. The low (near zero) R2* regions can be used for identifying regions of low susceptibility contrasts such as cerebrospinal fluid in the ventricles in the brain. As these regions have known susceptibility values, they can be used to serve zero references (to water) to generate absolute susceptibility values using a minimal variance regularization and to suppress streaking and shadowing artifacts.

After obtaining the susceptibility prior information and signal data noise property, the process 800 continues by estimating a magnetic susceptibility distribution of the subject based, at least in part, on the prior information and data noise property (step 860). As described above, estimating the susceptibility distribution of the subject can include determining a cost function corresponding to a susceptibility distribution, the magnetic field or the multiecho complex data. The cost function in some includes a data fidelity term based on data noise property expressing the relationship between tissue susceptibility and the magnetic field in an integral or differential form or expressing the relationships between tissue susceptibility and the multiecho complex data, and a regularization term expressing prior information. The estimated susceptibility distribution of the subject can be determined by identifying a particular susceptibility distribution that minimizes one or more of these cost functions described above, in some cases, this can be determined numerically.

After estimating a magnetic susceptibility distribution of the subject, the process 800 continues by generating one or more images of the subject based on the estimated susceptibility distribution of the subject (step 870). These images can be electronically displayed on a suitable display device (e.g., an LCD display device, LED display device, CRT display device, or other display device that can be configured to show images) and/or physically displayed on a suitable medium (e.g., printed, etched, painted, imprinted, or otherwise physically presented on a sheet or paper, plastic, or other material).

In some cases, the estimated susceptibility distribution of the subject can be visualized using a color scale, where each of several colors is mapped to a particular susceptibility value or range of susceptibility values. Accordingly, a two dimensional or three dimensional image can be generated, where each pixel or voxel of the image corresponds to a particular spatial location of the subject, and the color of that pixel of voxel depicts the susceptibility value of the subject at that location.

In some cases, the color scale can include a gradient of colors and/or a gradient of gray shades (e.g., a gray scale), in order to depict a range of susceptibility values. In some cases, for a color scale that includes a gradient of colors or a gradient of gray shades, one end of the gradient can correspond to the lowest susceptibility value in a particular window of susceptibility values (e.g., an arbitrarily selected window of values), and the other end of the gradient can correspond to the highest susceptibility value in the window of values. For instance, for gray scale that includes a gradient of gray shades between pure white and pure black, pure white can be used to indicate the highest susceptibility value in a particular arbitrary window of values, and pure black can be used to indicate the lowest susceptibility value in the window of values. Other relationships between a color/gray scale and susceptibility values is also possible. For example, in some cases, for gray scale that includes a gradient of gray shades between pure white and pure black, pure white can be used to indicate the lower susceptibility value in a particular arbitrary window of values, and pure black can be used to indicate the highest susceptibility value in the window of values. Although the examples are described in the context of gray scales, in practice, similar relationships between color scales and susceptibility values are also possible.

Susceptibility values and colors can be mapped linearly (e.g., such that each absolute change in susceptibility value corresponds to a proportional change in color), logarithmically (e.g., such that each exponential change in susceptibility value correspond to a linear change in color), or according to any other mapping. Although examples mapping between color scales and susceptibility values are described above, these are merely illustrative examples. In practice, other mappings are also possible, depending on the implementation.

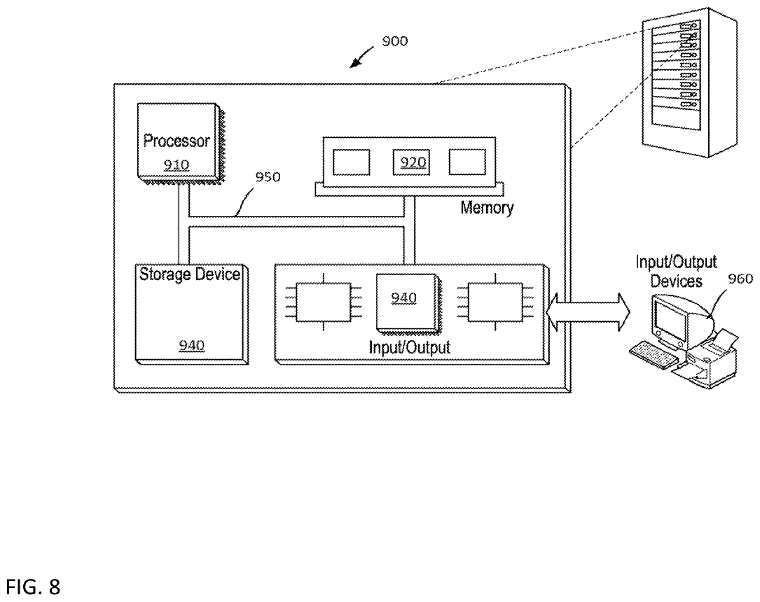

Implementations of the above described techniques can be performed using a computer system. FIG. 8 is a block diagram of an example computer system 900 that can be used, for example, to perform implementations of the process 800. In some implementations, the computer system 900 can be communicatively connected to another computer system (e.g., another computer system 900), such that it receives data (e.g., MRI datasets), and analyzes the received data using one or more of the techniques described above.

The system 900 includes a processor 910, a memory 920, a storage device 930, and an input/output device 940. Each of the components 910, 920, 930, and 940 can be interconnected, for example, using a system bus 950. The processor 910 is capable of processing instructions for execution within the system 900. In some implementations, the processor 910 is a single-threaded processor. In some implementations, the processor 910 is a multi-threaded processor. In some implementations, the processor 910 involves a graphic processing unit. In some implementations, the processor 910 is a quantum computer. The processor 910 is capable of processing instructions stored in the memory 920 or on the storage device 930. The processor 910 may execute operations such as performing one or more of the techniques described above.

The memory 920 stores information within the system 900. In some implementations, the memory 920 is a computer-readable medium. In some implementations, the memory 920 is a volatile memory unit. In some implementations, the memory 920 is a non-volatile memory unit.

The storage device 930 is capable of providing mass storage for the system 900. In some implementations, the storage device 930 is a non-transitory computer-readable medium. In various different implementations, the storage device 930 can include, for example, a hard disk device, an optical disk device, a solid-state drive, a flash drive, magnetic tape, or some other large capacity storage device. In some implementations, the storage device 930 may be a cloud storage device, e.g., a logical storage device including multiple physical storage devices distributed on a network and accessed using a network. In some examples, the storage device may store long-term data. The input/output device 940 provides input/output operations for the system 900. In some implementations, the input/output device 940 can include one or more of network interface devices, e.g., an Ethernet card, a serial communication device, e.g., an RS-232 port, and/or a wireless interface device, e.g., an 802.11 card (e.g. 802.11ax), a 3G wireless modem, a 4G wireless modem, a 5G wireless modem, etc. A network interface device allows the system 900 to communicate, for example, transmit and receive data. In some implementations, the input/output device can include driver devices configured to receive input data and send output data to other input/output devices, e.g., a keyboard, a mouse, a printer, a sensor (e.g., a sensor that measures component or system-related properties, a sensor that measures environmental-related properties, or other types of sensors), and a display device 960. In some implementations, mobile computing devices, mobile communication devices, and other devices can be used.

A computing system can be realized by instructions that upon execution cause one or more processing devices to carry out the processes and functions described above, for example, storing, maintaining, and displaying information. Such instructions can include, for example, interpreted instructions such as script instructions, or executable code, or other instructions stored in a computer readable medium. A computing system can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

Although an example processing system has been described in FIG. 8, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification, such as performing one or more of the above described processes, can be implemented as one or more computer program products, e.g., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “processing module” may encompass all apparatus, devices, and machines for processing data, including by way of example a programmable processor, a computer, or multiple processors or computers. A processing module can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM and DVD-ROM disks. The processor and the memory can be supplemented by, or incorporated in, special purpose logic circuitry. Sometimes a server is a general purpose computer, and sometimes it is a custom-tailored special purpose electronic device, and sometimes it is a combination of these things. Implementations can include a back end component, e.g., a data server, or a middleware component, e.g., an application server, or a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

Certain features that are described above in the context of separate implementations can also be implemented in combination in a single implementation. Conversely, features that are described in the context of a single implementation can be implemented in multiple implementations separately or in any sub-combinations.

The order in which operations are performed as described above can be altered. In certain circumstances, multitasking and parallel processing may be advantageous. The separation of system components in the implementations described above should not be understood as requiring such separation.

A number of embodiments of the invention have been described. Nevertheless, it will be understood that various modifications may be made without departing from the spirit and scope of the disclosure. Accordingly, other embodiments are within the scope of the following claims.

What is claimed is:

1. A method for generating one or more images of an object, the method comprising:
obtaining a multiecho complex magnetic resonance susceptibility imaging data collected by a magnetic resonance scanner, wherein the multiecho complex magnetic resonance susceptibility imaging data comprises magnitude and phase information regarding the object;
decomposing the object magnetic susceptibility sources into multiple compartments differentially affecting the multiecho complex magnetic resonance susceptibility imaging data;
determining a distribution of a susceptibility compartment in the object from processing the multiecho complex magnetic resonance susceptibility imaging data, wherein processing involves a deep neural network, wherein the deep neural network is trained on simulated multiecho complex magnetic resonance susceptibility imaging data, wherein the simulation comprises:
synthesizing susceptibility compartments according to an object configuration,
synthesizing multiecho complex magnetic resonance susceptibility imaging signal for the object configuration by summing signal contributions from all components in a voxel according to known physical laws, wherein the known physical laws include signal dephasing generated by magnetic susceptibility sources according to the dipole field,
generating a voxel value for a susceptibility compartment for the object configuration;
generating the one or more images of the object based on the determined distribution of the susceptibility compartment in the object; and
presenting, on a display device, the one or more images of the object.

2. The method of claim 1, wherein a susceptibility compartment involves deoxyheme iron in vasculature, and one image of the object generated is oxygen extraction fraction map.

3. The method of claim 1, wherein a susceptibility compartment is diamagnetic or paramagnetic.

4. The method of claim 1, wherein the known physical laws include water proton diffusion.

5. The method of claim 1, wherein the susceptibility compartments and the signal components within a voxel are represented in a digital twin.

6. A method for generating one or more images of an object, the method comprising:
obtaining a multiecho complex magnetic resonance susceptibility imaging data collected by a magnetic resonance scanner, wherein the multiecho complex magnetic resonance susceptibility imaging data comprises magnitude and phase information regarding the object;
decomposing the object magnetic susceptibility sources into at least two compartments differentially affecting the multiecho complex magnetic resonance susceptibility imaging data, wherein one compartment is paramagnetic;
determining a distribution of a susceptibility compartment in the object from processing the multiecho complex magnetic resonance susceptibility imaging data, wherein processing comprises
modeling the multiecho complex magnetic resonance susceptibility imaging data through summing signal contributions from all components in a voxel according to known physical laws, wherein the known physical laws include signal dephasing generated by magnetic susceptibility sources according to the dipole field, and
performing a spatial deconvolution to extract the susceptibility compartment;
generating the one or more images of the object based on the determined distribution of the magnetic source component in the object; and
presenting, on a display device, the one or more images of the object.

7. The method of claim 6, wherein the compartment being paramagnetic contains deoxyheme iron in vasculature and one image of the object is oxygen extraction fraction map.

8. The method of claim 6, wherein another compartment is myelin or diffuse paramagnetic iron.

9. The method of claim 6, wherein the susceptibility imaging data modeling takes a closed form and the processing involves an iterative optimization.

10. The method of claim 6, wherein the processing involves a deep neural network.

11. A method for determining a particle distribution in an object using magnetic resonance imaging, the method comprising
obtaining magnetic resonance imaging data from the object, wherein magnetic resonance imaging involves using a contrast agent and includes dynamic phase imaging;
determining a particle distribution in the patient from magnetic resonance imaging data, comprising
generating a vasculature from magnetic resonance imaging data, and
calculating tissue perfusion from dynamic phase imaging;
presenting, on a display device, the determined particle distribution.

12. The method of claim 11, wherein generating a vasculature involves a branching algorithm including capillaries as terminal branches and guidance from magnetic resonance imaging data.

13. The method of claim 11, wherein the contrast agent is a paramagnetic nanoparticle and magnetic resonance imaging includes pre-injection and equilibrium phase imaging.

14. The method of claim 11, wherein calculating tissue perfusion involves fitting the transport equation.

15. The method of claim 11, wherein the particle is the microsphere used in transarterial embolization, and the particle distribution is used to calculate the lung shunt fraction.

16. A system for generating one or more images of an object using magnetic resonance imaging, the system comprising a processor, a graphical output module communicatively coupled to the processor, an input module communicatively coupled to the processor, and a non-transitory computer storage medium encoded with a computer program, the program comprising instructions that when executed by processor cause the processor to perform operations comprising:
obtaining a multiecho complex magnetic resonance susceptibility imaging data collected by a magnetic resonance scanner, wherein the multiecho complex magnetic resonance susceptibility imaging data comprises magnitude and phase information regarding the object;
decomposing the object magnetic susceptibility sources into multiple compartments differentially affecting the multiecho complex magnetic resonance susceptibility imaging data;
determining a distribution of a susceptibility compartment in the object from processing the multiecho complex magnetic resonance susceptibility imaging data, wherein processing involves a deep neural network, wherein the deep neural network is trained on simulated multiecho complex magnetic resonance susceptibility imaging data, wherein the simulation comprises:
synthesizing susceptibility compartments according to an object configuration,
synthesizing multiecho complex magnetic resonance susceptibility imaging signal for the object configuration by summing signal contributions from all components in a voxel according to known physical laws, wherein the known physical laws include signal dephasing generated by magnetic susceptibility sources according to the dipole field,
generating a voxel value for a susceptibility compartment for the object configuration;
generating the one or more images of the object based on the determined distribution of the susceptibility compartment in the object; and
presenting, on a display device, the one or more images of the object.

17. A system for generating one or more images of an object using magnetic resonance imaging, the system comprising a processor, a graphical output module communicatively coupled to the processor, an input module communicatively coupled to the processor, and a non-transitory computer storage medium encoded with a computer program, the program comprising instructions that when executed by processor cause the processor to perform operations comprising:
obtaining a multiecho complex magnetic resonance susceptibility imaging data collected by a magnetic resonance scanner, wherein the multiecho complex magnetic resonance susceptibility imaging data comprises magnitude and phase information regarding the object;
decomposing the object magnetic susceptibility sources into at least two compartments differentially affecting the multiecho complex magnetic resonance susceptibility imaging data, wherein one compartment is paramagnetic;
determining a distribution of a susceptibility compartment in the object from processing the multiecho complex magnetic resonance susceptibility imaging data, wherein processing comprises
modeling the multiecho complex magnetic resonance susceptibility imaging data through summing signal contributions from all components in a voxel according to known physical laws, wherein the known physical laws include signal dephasing generated by magnetic susceptibility sources according to the dipole field, and
performing a spatial deconvolution to extract the susceptibility compartment;
generating the one or more images of the object based on the determined distribution of the magnetic source component in the object; and
presenting, on a display device, the one or more images of the object.

18. A system for generating one or more images of an object using magnetic resonance imaging, the system comprising a processor, a graphical output module communicatively coupled to the processor, an input module communicatively coupled to the processor, and a non-transitory computer storage medium encoded with a computer program, the program comprising instructions that when executed by processor cause the processor to perform operations comprising
obtaining magnetic resonance imaging data from the patient, wherein magnetic resonance imaging involves using a contrast agent and includes dynamic phase imaging;
determining a particle distribution in the patient from magnetic resonance imaging data, comprising
generating a vasculature from magnetic resonance imaging data, and
calculating tissue perfusion from dynamic phase imaging;
presenting, on a display device, the determined particle distribution.
